(19) United States
(12) Patent Application Publication
(10) Pub. No.: US 2023/0112943 A1
(43) Pub. Date: Apr. 13, 2023

(54) TOPICALLY ADMINISTRABLE LYZATE OF DEDIFFERENTIATED CELLS OF THE PLANT HELICHRYSUM STOECHAS FOR MOISTURIZING THE SKIN

(71) Applicants: SOCIETE D'EXPLOITATION DE PRODUITS POUR LES INDUSTRIES CHIMIQUES - SEPPIC, PARIS CEDEX 07 (FR); BIOTECHMARINE, Quemper Guezennec (FR)

(72) Inventors: Erwan LE GELEBART, QUEMPER GUÉZENNEC (FR); Cécile BIZE, CASTRES (FR)

(21) Appl. No.: 17/800,193

(22) PCT Filed: Feb. 15, 2021
(86) PCT No.: PCT/EP2021/053646

§ 371 Date: Aug. 16, 2022

(30) Foreign Application Priority Data
Feb. 17, 2020 (FR) FR2001547

(51) Int. Cl. A61K 8/9794; A61K 8/73; A61K 8/06; A61K 8/92; A61Q 19/00

(52) U.S. Cl. CPC A61K 8/9794; A61K 8/731; A61K 8/732; A61K 8/062; A61K 8/92; A61Q 19/007; A61Q 19/001

(57) ABSTRACT

Topically administrable lyzate of dedifferentiated cells of the plant Helichrysum stoechas for improving the moisturizing of the skin and/or the scalp and/or the lips.

### CROSS-REFERENCE TO RELATED APPLICATIONS

This application is the U.S. national phase of International Application No. PCT/EP2021/053646 filed Feb. 15, 2021 which designated the U.S. and claims priority to French Patent Application No. 2001547 filed Feb. 17, 2020, the entire contents of each of which are hereby incorporated by reference.

### BACKGROUND OF THE INVENTION

### Field of the Invention

The present invention relates to a topically administrable lyzate of dedifferentiated cells of the plant Helichrysum stoechas for moisturizing human skin and/or scalp and/or lips, more particularly dry, reactive and/or sensitive skin.

### Description of the Related Art

Moisturization is a property of the skin involving complex mechanisms that are still not all known to date. These mechanisms are located in all the layers of the skin.

The skin constitutes the interface between the human internal medium (or human body) and the external environment. As a result, and with the flora which covers and inhabits it, the skin notably has the function of performing a mission of protection of the human body, by forming a genuine barrier which is vital. In particular, the skin makes it possible to combat dehydration by limiting the diffusion of water out of the body.

Due to its interface position with the external environment, the skin is subjected to numerous daily stresses, for instance contact with clothing, changes in temperature, exposure to the ultraviolet rays of sunlight, changes in humidity levels, contact with certain irritant chemicals or contact with chemicals regarded as pollutants.
The skin is composed of layers of different tissues:
   the epidermis, composed of keratinocytes, is its outermost part, followed by
   the dermis, which is a connective tissue mainly composed of fibroblasts and of extracellular matrix, and
   the hypodermis, consisting of adipocytes, which is the deepest part and the part furthest from the external environment.
The skin performs various functions in the interest of the entire system which it shelters, among which are included the following:
   a mechanical barrier function in order to guarantee the integrity of the internal medium of the body,
   an emunctorial function directed toward secreting sweat based on water, salts and acidic waste,
   a function of regulating the temperature of the body, and contains many other regulatory mechanisms, for instance its mechanism of adaptation and of protection against ultraviolet radiation (adaptive pigment coloring via the production of melanin), for instance a system for immune monitoring by the presence of macrophages or dendritic cells.

Human skin also constitutes the first image seen by others.

Consequently, improving its appearance is a matter of constant concern for humans. The skin is the reflection of a state of well-being, often associated with youthfulness, and, conversely, with a state of tiredness and/or aging. As a result, preserving and improving the state of the outermost layer of the skin, namely the epidermis, is a major focus for the research conducted by the cosmetics industries.

At the periphery of the epidermis is an upper cornified layer known as the stratum corneum, which is the first layer of the epidermis to be subjected to stresses of external origin, such as variations in external weather conditions (temperature, hygrometry, ultraviolet radiation, pollutants) or mechanical stresses.

This is why improving the appearance or maintaining the satisfactory appearance of human skin consists notably in maintaining optimum and satisfactory moisturization of the stratum corneum. This also makes it possible to avoid esthetic and physiological drawbacks associated with dryness of the skin. Moreover, dry or very dry skin is a chronic condition that can be linked to a genetic dysfunction: it is a skin type, just as there is the greasy skin type, the combination skin type, etc.

However, beyond constitutional dryness, there are also states of skin dryness that are triggered by external, climatic or environmental factors, caused by skin pathologies (atopic eczema, psoriasis, etc.), general diseases (thyroid, diabetes, nutritional deficiency) and/or medical treatments. The skin no longer fulfils its barrier function effectively: it becomes dehydrated. Thus, skin dryness is caused by weakening or impairment of the skin's barrier function.
Dry skin is mainly characterized by:
   light desquamation;
   the presence of roughness;
   the presence of redness;
   the appearance of chapping or micro-cracks;
   loss of elasticity;
   the development of skin sensitization, which is manifested by slight tautness, tingling, itching and pain.

Consequently, ensuring good moisturization of the upper layers of the epidermis, the stratum corneum, allows:
maintenance of the elasticity of the skin;
protection of the skin against external damage;
good functioning of the hydrolytic enzymes involved in the mechanism of desquamation in order to ensure optimal renewal of the epidermis;
optimal contribution toward the skin's barrier function.

Sensitive skin is defined by a specific reactivity of the skin.

This cutaneous reactivity is conventionally reflected by the manifestation of signs of discomfort, such as redness, in response to an individual coming into contact with a triggering factor, which may have diverse origins.

It was recently defined as follows: “a syndrome defined by the occurrence of unpleasant sensations (stinging, burning, pain, pruritus and tingling sensations) in response to stimuli that normally should not provoke such sensations. These sensations cannot be explained by lesions attributable to any skin disease. The skin may appear normal or may be accompanied by erythema. Sensitive skin can affect all parts of the body, and in particular the face”. Environmental factors such as exposure to ultraviolet or infrared rays, and/or air pollution, and/or sudden temperature variations and/or wind, or lifestyle (dietary habits or application of cosmetic products on the skin surface), or physiological factors such as stress or endogenous hormones, have been recognized as being able to induce or aggravate the symptoms of sensitive skin. Two main reasons can explain the symptoms of sensitive skin: firstly, an increase in the permeability of the stratum corneum and, secondly, excessive secretion of certain neurotransmitters by the endings of the superficial nerves and proinflammatory cytokines.

For the purposes of the present invention, the term “sensitive skin” covers irritable skin and intolerant skin.

Intolerant skin is skin that reacts with sensations of heating, tautness, tingling and/or redness, to various factors such as the application of cosmetic or dermatological products or soap. In general, these signs are associated with erythema and with hyperseborrheic or acneic skin, or even rosaceiform skin, with or without dry patches.

Irritable skin is skin that reacts with pruritus, i.e. with itching or stinging, to various factors such as the environment, emotions, foods, the wind, friction, shaving, hard water with a high calcium concentration, temperature variations, humidity or wool.
Based on this, a problem that arises is that of providing a compound for moisturizing the skin and/or the scalp.

### SUMMARY OF THE INVENTION

One solution according to the present invention is a topically administrable lyzate (Ly) of dedifferentiated cells of the plant Helichrysum stoechas for improving the moisturizing of the skin and/or the scalp and/or the lips.

The term “topically administrable” means that the lyzate is formulated to be topically administrable.

The lyzate (Ly) of dedifferentiated cells of the plant Helichrysum stoechas will be the active principle for improving the moisturization of the skin and/or the scalp and/or the lips.

For the purposes of the present invention, an improvement in the moisturization of the skin, and more particularly of the stratum corneum, may be imagined by placing the skin in contact with compounds which have a “moisturizing effect”.

For the purposes of the present invention, the term “moisturizing effect” means:
a reduction in the transepidermal water loss, and/or
a decrease in the intercellular spaces between the keratinocytes of the basal layer of the epidermis, and/or
an increase in the thickness of the stratum corneum, and/or
restoration or improvement of the barrier function, and/or
an increase in lacunar zones, and/or
an increase in the degree of moisturization of the stratum corneum, resulting from the topical application of a chemical substance or composition to the surface of the skin to be treated.

According to a particular aspect, the present invention relates to a topically administrable lyzate (Ly) of dedifferentiated cells of the plant Helichrysum stoechas for preventing or slowing down the appearance of the external signs of aging of human skin, for instance the appearance of wrinkles, fine lines, impairment of the microrelief, lack of elasticity and/or tonicity, and lack of density and/or firmness of human skin.

Indeed, there is a link between skin aging and moisturization. Water is an essential component of the skin and the scalp. If the epidermis is well hydrated, full of water, it will have a plump, smooth appearance. Conversely, dehydration has the opposite effect on its quality, notably by the appearance of wrinkles, bags under the eyes and a gray complexion. Dehydratation is one of the natural consequences of aging. The epidermis has the ability to uptake moisture and maintain optimum moisturization. However, this capacity decreases with age. The aging of skin cells thus causes a decrease in the proportion of hyaluronic acid, whose role is to “trap” water molecules in the epidermis. The decrease in hormonal secretion that occurs at about the age of 50 adds to this. Thus, the skin gradually loses its level of moisturization.

Moreover, the lyzate according to the invention can be administered topically to moisturize the skin after shaving the face or to moisturize the scalp after cleansing and/or treating the scalp.

Preferably, the lyzate (Ly) is derived from the high pressure homogenization of the culture of dedifferentiated cells of the plant Helichrysum stoechas.

The process for obtaining the lyzate of dedifferentiated cells of the plant Helichrysum stoechas according to the invention will preferably comprise the following steps:
a) Preparation of the sterile sample of the plant Helichrysum stoechas
b) Callogenesis
c) Suspending
d) Selection of a fine suspension
e) Biomass production
f) High-pressure homogenization of the culture
g) Stabilization of the culture lyzate of dedifferentiated cells.

From step e), steps a), b), c) and d) no longer need to be repeated since it leads to the culture of dedifferentiated cells which can be used indefinitely to produce biomass provided that the physiological needs of the cells are respected.

Steps b), c), d) and e) must be performed under sterile conditions in order to keep the culture under axenic conditions.

The steps are described in detail as follows:
a) Preparation of the sterile sample of Helichrysum stoechas

A few leaves of Helichrysum stoechas are removed, and these leaves are cut into fragments of a few millimeters. The fragments of leaves are sterilized by immersion in successive baths containing:
a mixture of 70% ethanol containing a detergent, such as Tween 80, in order to improve the effectiveness of the sterilization for a period of 5 minutes, and then in
a sodium hypochlorite solution at 1% by mass for a period of 5 minutes.

The fragments of the leaves thus sterilized are then rinsed by immersion in a bath of sterile distilled water three times in succession.

This step a) may also be performed using any other part of the plant (roots, stems, meristems, flowers, etc.). The sterilization may also be applied to the seeds, without the cutting phase.
b) Callogenesis

Pieces of sterilized leaves obtained in the preceding step are deposited on the solid callogenesis medium (the composition of which is described in Table 1), which is then incubated for a period of three weeks in a thermostatic chamber at a temperature of greater than or equal to 20 and less than or equal to 25° C. in the light or dark.

TABLE 1

| Compounds | Concentration (mg/L) |
|---|---|
| Glucose or sucrose | 30 000 |
| Naphthaleneacetic acid | 1 |
| 6-Benzylaminopurine | 0.5 |
| Agar | 10 000 |
| KNO₃ | 2500 |
| (NH₄)₂SO₄ | 134 |
| CaCl₂•2H₂O | 150 |
| NaH₂PO₄•2H₂O | 150 |
| MgSO₄•7H₂O | 250 |
| MnSO₄•H₂O | 16.9 |
| ZnSO₄•7H₂O | 8.6 |
| H₃BO₃ | 6.2 |
| KI | 0.83 |
| Na₂MoO₄•2H₂O | 0.25 |
| CuSO₄•5H₂O | 0.025 |
| FeSO₄•7H₂O | 27.8 |
| Na₂EDTA•2H₂O | 37.3 |
| Myo-inositol | 100 |
| Nicotinic acid | 1 |
| Pantothenic acid | 1 |
| Biotin | 0.01 |
| Pyridoxine | 1 |
| Thiamine | 1 |

### Composition of the Callogenesis Medium

a) Suspending the Dedifferentiated Cells

The callus formed during step b) is withdrawn and placed in a liquid suspending medium (the composition of which is shown in table 2). For the purposes of the present invention, the term “callus” means a cluster of dedifferentiated cells.

The pieces of callus in suspension are cultured in a thermostatically controlled chamber for a period of 7 days to 21 days at a temperature of greater than or equal to 20° C. and of less than or equal to 25° C. in the light or in the dark, under orbital-type mechanical agitation, at a stirring rotation of between 25 rpm and 200 rpm (more specifically at a rotation speed of 100 rpm for an orbital diameter of 5 cm).

TABLE 2

| Compounds | Concentration (mg/L) |
|---|---|
| Glucose or sucrose | 30 000 |
| Naphthaleneacetic acid | 1 |
| 6-Benzylaminopurine | 0.5 |
| KNO₃ | 2500 |
| (NH4)₂SO₄ | 134 |
| CaCl₂•2H₂O | 150 |
| NaH₂PO₄•2H₂O | 150 |
| MgSO₄•7H₂O | 250 |
| MnSO₄•H₂O | 16.9 |
| ZnSO₄•7H₂O | 8.6 |
| H₃BO₃ | 6.2 |
| KI | 0.83 |
| Na₂MoO₄•2H₂O | 0.25 |
| CuSO₄•5H₂O | 0.025 |
| FeSO₄•7H₂O | 27.8 |
| Na₂EDTA•2H₂O | 37.3 |
| Myo-inositol | 100 |
| Nicotinic acid | 1 |
| Pantothenic acid | 1 |
| Biotin | 0.01 |
| Pyridoxine | 1 |
| Thiamine | 1 |

Composition of the callus suspension medium formed in step b).

b) Selection of a Fine Suspension

Take between ⅕ and ⅓ of the volume of culture containing dedifferentiated cells in fine suspension obtained on conclusion of step c.

Place this volume of fine suspension in a volume of fresh suspending medium, three times greater than the volume of fine suspension.

This new suspension is cultured in a thermostatically controlled chamber for a period of 7 to 21 days (the end of the culture period is dictated by the observation of nutrient deficiency) at a temperature greater than or equal to 20 and less than or equal to −25° C. in the light, under orbital-type mechanical agitation, at a rotational speed of between 25 rpm and 200 rpm (more precisely, at a rotational speed of 100 rpm for an orbital diameter of 5 cm). For the purposes of the present invention, the term “nutrient deficiency” means the observation of a content of less than 5% of the amount of nutrient initially introduced into the culture medium.

a) Production of Biomass Comprising Dedifferentiated Cells and Culture Medium

Withdraw a volume of the fine suspension obtained during step d) and place it in a volume of fresh suspending medium, as described in table 2, which is three times greater than the withdrawn volume of fine suspension obtained on conclusion of step d).

This new suspension is cultured in a thermostatically controlled chamber for a period of 7 to 21 days (the end of the culture period is dictated by nutrient deficiency), at a temperature greater than or equal to 20 and less than or equal to 25° C., in the light or in the dark, under orbital-type mechanical agitation, at a rotation speed of between 25 rpm and 200 rpm (more precisely at 100 rpm for an orbital diameter of 5 cm). This step can be performed, up to volumes of less than or equal to 5 liters, in conical flasks, then, for volumes of greater than 5 liters and less than or equal to 1000 liters, in bioreactors (of “wave reactor”, “stirred tank reactor” or conical flask type) (this list not being exhaustive).

b) High Pressure Homogenization of the Culture: Production of the Culture Lyzate of Dedifferentiated Cells.

A fraction of the volume of culture obtained on conclusion of step e) is taken to be lyzed using a high-pressure homogenizer, the pressure of which may be set at a value greater than or equal to 100 and less than or equal to 1000 bar (preferably for the present invention, at a pressure equal to 500 bar). The dedifferentiated cells present in the culture passed through the high-pressure homogenizer are lyzed, and their contents are released into the culture medium. The homogenization efficiency (which is reflected by the observation of cell debris indicating lysis of the dedifferentiated cells) can be readily checked by microscopic observation of the suspension.

c) Stabilization of the Culture Lyzate of Dedifferentiated Cells

The culture lyzate of dedifferentiated cells which is collected on conclusion of step f) is acidified, by adding a mineral acid or an organic acid, to achieve a pH value of greater than or equal to 4 and of less than or equal to 4.5, so as to stabilize its color. This lyzate contains pieces of cells which are insoluble in the medium and which are liable to settle out, and to constitute an obstacle to the commercialization of the product. The addition of a thickener and/or gelling agent, in a proportion greater than or equal to 0.1% by mass and less than or equal to 2% (more preferentially a mixture of xanthan gum and acacia gum, in mass proportions of 40-45% and 48-55% and in an amount of 0.8% by mass, such as the mixture of xanthan gum and acacia gum sold under the brand name Solagum™ AX) makes it possible to obtain a suspension which does not produce sediment over time.

A lyzate of dedifferentiated cells can be stabilized from a microbiological and physicochemical viewpoint by various means:
by pasteurization (example: UHT pasteurization),
by modification of the pH (example: acidification pH<5),
by addition of an agent for reducing the availability of water (example: glycerol),
by addition of preserving agents (example: sodium benzoate, potassium sorbate).

In the present case, it is preferred to stabilize the lyzate by adding a mixture of glycerol, potassium sorbate and sodium benzoate.

The proportions of the various ingredients are as follows, per 100% by mass:
60% to 70% by mass of HPH lyzate of the Helichrysum stoechas dedifferentiated cell culture obtained in step f (95% to 99.5% by mass of water and 0.5% to 5% by mass of solids)
30% to 40% by mass of glycerol
0.1% to 2% by mass of Solagum™ AX (mixture of acacia gum and xanthan gum)
0.1% to 2% of potassium sorbate
0.1% to 2% of sodium benzoate.

The stabilized culture lyzate of dedifferentiated cells can be stored or be directly incorporated in a cosmetic formulation in an amount of 0.1% to 3% by mass (preferentially 1% by mass).

Any kind of thickener can act as a stabilizer, for example use is made in the present case of a solvent of polar nature, preferably glycerol, but other polyols could be used.

It should be noted that there are many differences between the whole plant and the dedifferentiated cells. To show these differences, a comparison was made. The comparison between the whole plant and the dedifferentiated cells was able to be made by means of comparing ethanolic extracts analyzed by high pressure liquid chromatography (HPLC). The samples are analyzed on a Kinetex C18 150*4.6 mm HPLC column with a flow rate of 0.8 ml/min. The solvents are as follows:
A: isopropanol containing 0.05% TFA,
B: acetonitrile containing 0.05% TFA,
C: water containing 0.05% TFA.

The gradient is as follows:

| t (min) | % A | % B | % C |
|---|---|---|---|
| 0 | 0 | 2 | 98 |
| 5 | 0 | 2 | 98 |
| 20 | 0 | 100 | 0 |
| 25 | 0 | 100 | 0 |
| 35 | 80 | 20 | 0 |
| 40 | 80 | 20 | 0 |
| 45 | 0 | 100 | 0 |
| 50 | 0 | 2 | 98 |

This analysis was performed with detection of CAD (Charged Aerosol Detection) type. The results are obtained from a chromatogram of an ethanolic extract of aerial parts of Helichrysum stoechas with CAD detection according to the HPLC C18_screening method and a chromatogram of an ethanolic extract of dedifferentiated cells of Helichrysum stoechas with CAD detection according to the HPLC C18_screening method.

The results obtained clearly show a difference between the two chromatograms. The chromatogram of the cell extract is much richer in the region between the retention times between 25 minutes and 40 minutes, which corresponds to the elution times of the most nonpolar molecules.

A subject of the present invention is also a composition (C1) for topical use which is in the form of a gel and which comprises, per 100% of its mass:
from 95% to 99.5% by mass, preferably from 97% to 99.5% by mass, even more preferably from 97.5% to 99.5% by mass of lyzate (Ly) as defined previously and preferably obtained by performing a process comprising steps a) to f) as described above,
from 0.5% to 5% by mass, preferably from 0.5% to 3% by mass, even more preferentially from 0.5% to 2.5% by mass, of at least one thickener and/or gelling agent.

Preferably, the gelling agents and/or thickeners are chosen from polysaccharides, cellulose and cellulose derivatives, starches and linear or branched or crosslinked polymers of polyelectrolyte type.

This composition (C1) may be administered topically to improve the moisturization of the skin and/or the scalp and/or the lips.

In the context of the present invention, the term “gel” denotes a chemical composition which is initially in liquid form and which, after addition of gelling and/or thickening substances, is transformed into a structured state, which does not flow. A gel as defined is considered as an intermediate state between the solid state and the liquid state, and consists of a three-dimensional network within the liquid, which is the result of chemical or physical bonds.

For the purposes of the present invention, the term “gelling agent” means a chemical compound or a mixture of chemical compounds which transforms a liquid medium into a gel.

For the purposes of the present invention, the term “thickener” means a chemical compound or a mixture of chemical compounds which increases the viscosity of the medium into which it is introduced.

The term “for topical use” used in the definition of composition (C₁) as described above means that said composition is formulated to enable its application to the skin, the hair, the scalp, the nails, the lips, mucous membranes, the eyelashes or the eyebrows, whether it is a direct application in the case of a cosmetic formulation or an indirect application, for example in the case of a bodycare product in the form of a textile or paper wipe, or of sanitary products intended to be in contact with the skin, the hair, the scalp, the nails, the lips, mucous membranes, the eyelashes or the eyebrows.

In the context of the present invention, the term “polysaccharides” denotes saccharide polymers. The IUPAC definition of saccharides designates monosaccharides, compounds of monosaccharides per se and derivatives thereof, obtained either by reduction of a carbonyl group, or by oxidation of one or more hydroxyl functions, or by the replacement of one or more hydroxyl functions with a hydrogen atom, an amine group, a phosphate function, or a sulfate function. The polysaccharides most commonly used for preparing industrial food, cosmetic or pharmaceutical compositions predominantly consist of monosaccharides, such as glucose, galactose, mannose or of monosaccharide derivatives for which the hydroxyl function of the terminal carbon has been oxidized to a carboxyl function. Two distinct groups may be distinguished among the polysaccharides: polysaccharides consisting solely of monosaccharides (or poly-monosaccharides) and polysaccharides consisting of monosaccharide derivatives.

According to a particular aspect, the gelling agents and/or thickeners present in the aqueous composition (C1) that is the subject of the present invention are chosen from polysaccharides consisting solely of monosaccharides (or poly-monosaccharides).

Among the polysaccharides composed solely of monosaccharides, a distinction may be made between glucans, which are glucose homopolymers that are very abundant in nature, glucomannoglycans, xyloglycans and galactomannans, which are polymers whose main chain consists of D-mannose units, connected together via 3-1,4 bonds, and on which D-galactose units are grafted laterally via α-1,6 bonds.

Galactomannans are present in several plant species, and more particularly in the leguminous species in which they constitute the albumen of seeds. Depending on their plant origin, the degree of substitution (DS) of the D-galactose units on the D-mannose main chain of galactomannans ranges between 0 and 1:
galactomannans originating from cassia gum have a degree of substitution (DS) of approximately ⅕, meaning the lateral grafting of one D-galactose unit every 5 D-mannose units present on the main chain of the polysaccharide;
galactomannans originating from locust bean gum have a degree of substitution (DS) of approximately ¼, meaning the lateral grafting of one D-galactose unit every 4 D-mannose units present on the main chain of the polysaccharide;
galactomannans originating from tara gum have a degree of substitution (DS) of approximately ⅓, meaning the lateral grafting of one D-galactose unit every 3 D-mannose units present on the main chain of the polysaccharide;
galactomannans originating from guar gum have a degree of substitution (DS) of approximately ½, meaning the lateral grafting of one D-galactose unit every 2 D-mannose units present on the main chain of the polysaccharide;
galactomannans originating from fenugreek gum have a degree of substitution (DS) of approximately 1/1, meaning the lateral grafting of one D-galactose unit for virtually every D-mannose unit present on the main chain of the polysaccharide.

According to a more particular aspect, the gelling agents and/or thickeners present in the aqueous composition (C1) that is the subject of the present invention are chosen from polysaccharides consisting solely of monosaccharides (or poly-monosaccharides) included in the group consisting of galactomannan originating from tara gum, galactomannan originating from guar gum and galactomannan originating from locust bean gum.

According to another particular aspect, the gelling agents and/or thickeners present in the aqueous composition (C₁) that is the subject of the present invention are chosen from polysaccharides consisting of monosaccharide derivatives. Among the polysaccharides consisting of monosaccharide derivatives, the following may be distinguished:
sulfated galactans, which are polymers of galactose which may have pendent sulfate-ester groups, represented notably by algal polysaccharides such as carrageenans and agar;
uronans, which are polymers of uronic acids such as algins and pectins;
heteropolymers of monosaccharides and uronic acids: often of complex composition, these polymers are found notably in sap exudates (for instance acacia gum exudate and karaya gum exudate), but they are also produced by microorganisms, for instance xanthan gum and gellan gum;
glucosaminoglycans which are polysaccharides formed from a glucose derived by replacing its C-2 hydroxyl with an amine (referred to as 2-amino-2-deoxy-D-glucose or, more simply, glucosamine). The amine function may moreover be acetylated. Among the hydrocolloids in this class are chitosan, formed solely of glucosamine units, and hyaluronan, the repeating unit of which is a dimer of glucosamine and glucuronic acid.

Xanthan gum (G_{X}) has in recent decades become the microbial polysaccharide that is the most widely used in industry. Xanthan is a polysaccharide synthesized by bacteria of the genus Xanthomonas and, commercially, only the species X. campestris is used. The main chain of (G_{X}) is identical to that of cellulose, i.e. it is formed from 3-D-glucose units connected together via carbons 1 and 4. There is one branched trisaccharide every two glucose units in the main chain, in a regular alternating manner; each branch consisting of a trisaccharide composed of two mannoses and one glucuronic acid, of the type: β-D-Manp-(1→4)-B-D-GlcAp-(1→2)-A-D-Manp-(1→3) (I. Capron et al., “About the native and renaturated conformation of xanthan exopolysaccharide”, 1997). Xanthan gum (G_{X}) is available in the form of a sodium, potassium or calcium salt.

Acacia gum is a complex, branched polysaccharide whose main chain consists of 3-D-galactose units connected together via carbons 1 and 3. The chains branched to the main chain consist of 3-D-galactose units connected together via carbons 1 and 6, also bearing α-arabinose units, and to a lesser extent β-glucoronosyl units. Both the main chain and the pendent chains contain α-L-arabinosyl, α-L-rhamnopyranosyl, β-D-glucuronopyranosyl and 4-O-methyl-β-D-glucuronopyranosyl units.

According to a more particular aspect, the gelling agents and/or thickeners present in the aqueous composition (C₁) that is the subject of the present invention are polysaccharides consisting of monosaccharide derivatives chosen from the elements of the group consisting of carrageenans, agar, algins, pectins, acacia gum exudate, karaya gum exudate, xanthan gum, gellan gum, chitosan and hyaluronan, and/or mixtures thereof.

According to an even more particular aspect, the gelling agents and/or thickeners present in the aqueous composition (C₁) that is the subject of the present invention are polysaccharides consisting of monosaccharide derivatives chosen from the elements of the group consisting of acacia gum exudate, karaya gum exudate and xanthan gum, and/or mixtures thereof.

According to an even more particular aspect, the gelling agents and/or thickeners present in the aqueous composition (C₁) that is the subject of the present invention are polysaccharides consisting of monosaccharide derivatives chosen from the elements of the group consisting of acacia gum exudate, xanthan gum, the mixture of xanthan gum (G_{X}) and acacia gum exudate (G_{A}) used in a mass ratio between the xanthan gum (G_{X}) and the acacia gum exudate (G_{A}) of greater than or equal to ⅓ and less than or equal to 3/1, sold notably by the company SEPPIC under the brand name Solagum™ AX.

According to a particular aspect, the gelling agents and/or thickeners present in the aqueous composition (C₁) that is the subject of the present invention are chosen from cellulose and cellulose derivatives.

In the context of the present invention, the term “cellulose” denotes a polysaccharide consisting of a linear chain of D-glucose molecules, the average molecular mass of which is at least 10 000 g·mol-1, more particularly at least 15 000 g·mol-1, more particularly at least 17 000 g·mol-1, even more particularly at least 20 000 g·mol-1 and even more particularly at least 25 000 g·mol-1.

According to a more particular aspect, the gelling agents and/or thickeners present in the aqueous composition (C₁) that is the subject of the present invention are chosen from cellulose derivatives.

In the context of the present invention, the term “cellulose derivatives” denotes the elements of the group consisting of hydroxyethyl cellulose, methyl cellulose, ethyl cellulose, methyl hydroxyethyl cellulose, methyl hydroxypropyl cellulose, hydroxypropyl cellulose, the sodium salt of carboxymethyl cellulose and cellulose dihydroxypropyl ether.

In the context of the present invention, the term “starch” denotes a mixture of amylose and amylopectin, and more particularly the elements of the group consisting of corn starch, wheat starch, potato starch and cassava starch.

According to a particular aspect, the term “linear or branched or crosslinked polymers of polyelectrolyte type” denotes, for the purposes of the present invention:
crosslinked synthetic anionic copolymers based on methacrylic acid or acrylic acid, or esters of methacrylic acid or of acrylic acid, which are optionally hydrophobically modified, prepared by direct emulsion polymerization. These synthetic anionic copolymers are known, respectively, to a person skilled in the art under the names “Alkaline Swellable Emulsion” (or “ASE”) and “Hydrophobically Alkaline Swellable Emulsion” (or “HASE”). Thickening agents of HASE type are described in the international patent application published under the number WO 02/34793 A2;
crosslinked or branched synthetic anionic polyelectrolytes, which are crosslinked and/or branched homopolymers or copolymers of water-soluble unsaturated monomers, such as acrylic acid and/or derivatives thereof, methacrylic acid and/or derivatives thereof, acrylamide and/or derivatives thereof, 2-acrylamido-2-methylpropanesulfonic acid and/or salts thereof, N-vinylpyrrolidone, vinyl alcohol and/or derivatives thereof. These crosslinked or branched synthetic anionic polyelectrolytes are in the form of reverse latices, obtained by reverse emulsion radical polymerization, or in the form of powders, obtained by precipitating polymerization, or by atomization of reverse latices.

According to a particular aspect, the gelling agents and/or thickeners present in the aqueous composition (C₁) that is the subject of the present invention are chosen from linear or branched or crosslinked polyelectrolytes, obtained from the radical polymerization of at least one monomer selected from the elements of the group consisting of acrylic acid and/or the sodium salt thereof, methacrylic acid and/or the sodium salt thereof, 2-hydroxyethyl acrylate, 2-hydroxyethyl methacrylate, acrylamide, N,N-dimethylacrylamide, N-isopropylacrylamide, 2-acrylamido-2-methylpropanesulfonic acid and/or the sodium or the potassium salt thereof, N-vinylpyrrolidone, at least one monomer of formula (I):

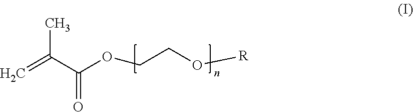

in which R represents a linear or branched alkyl radical including from 8 to 20 carbon atoms and n represents an integer greater than or equal to 0 and less than or equal to 20. Said radical polymerization is performed in the presence of a crosslinking agent chosen from polyethylenic monomers comprising at least two ethylenic functions, and more particularly chosen from the elements of the group consisting of ethylene glycol dimethacrylate, tetraallyloxyethane, ethylene glycol diacrylate, diallylurea, triallylamine, trimethylolpropane triacrylate and methylenebis(acrylamide), or a mixture of these compounds.

According to a particular aspect, the gelling agents and/or thickeners present in the aqueous composition (C₁) that is the subject of the present invention are chosen from the elements of the group consisting of:
partially or totally salified acrylic acid homopolymer, crosslinked with triallylamine and/or with trimethylolpropane triacrylate and/or with methylenebis(acrylamide),
the homopolymer of the sodium salt of 2-acrylamido-2-methylpropanesulfonic acid, crosslinked with triallylamine and/or with trimethylolpropane triacrylate and/or with methylenebis(acrylamide),
the copolymer of the sodium salt of 2-acrylamido-2-methylpropanesulfonic acid and of partially or totally salified acrylic acid, crosslinked with triallylamine and/or with trimethylolpropane triacrylate and/or with methylenebis(acrylamide),
the copolymer of the sodium salt of 2-acrylamido-2-methylpropanesulfonic acid and of 2-hydroxyethyl acrylate, crosslinked with triallylamine and/or with trimethylolpropane triacrylate and/or with methylenebis(acrylamide),
the copolymer of the sodium salt of 2-acrylamido-2-methylpropanesulfonic acid and of acrylamide, crosslinked with triallylamine and/or with trimethylolpropane triacrylate and/or with methylenebis(acrylamide),
the terpolymer of the sodium salt of 2-acrylamido-2-methylpropanesulfonic acid, of acrylamide and of partially or totally salified acrylic acid, crosslinked with triallylamine and/or with trimethylolpropane triacrylate and/or with methylenebis(acrylamide),
the terpolymer of the sodium salt of 2-acrylamido-2-methylpropanesulfonic acid, of N,N-dimethylacrylamide and of partially or totally salified acrylic acid, crosslinked with triallylamine and/or with trimethylolpropane triacrylate and/or with methylenebis(acrylamide),
a terpolymer, crosslinked with trimethylolpropane triacrylate and/or triallylamine and/or methylenebis(acrylamide), of 2-methyl-2-[(1-oxo-2-propenyl)amino]-1-propanesulfonic acid partially or completely salified in sodium salt or ammonium salt form, in a molar proportion of greater than or equal to 60% and of less than or equal to 80%, of N,N-dimethylacrylamide, in a molar proportion of greater than or equal to 15% and of less than or equal to 39.5%, and tetraethoxylated lauryl methacrylate, in a molar proportion of greater than or equal to 0.5% and of less than or equal to 5%,
a tetrapolymer, crosslinked with trimethylolpropane triacrylate and/or triallylamine and/or methylenebis(acrylamide), of 2-methyl-2-[(1-oxo-2-propenyl)amino]-1-propanesulfonic acid partially or completely salified in sodium salt or ammonium salt form, in a molar proportion of greater than or equal to 60% and of less than or equal to 80%, of N,N-dimethylacrylamide, in a molar proportion of greater than or equal to 15% and of less than or equal to 39%, of lauryl methacrylate, in a molar proportion of greater than or equal to 0.5% and of less than or equal to 2.5%, and of stearyl methacrylate, in a molar proportion of greater than or equal to 0.5% and of less than or equal to 2.5%.

According to a more particular aspect, the gelling agents and/or thickeners present in the aqueous composition (C₁) that is a subject of the present invention are chosen from the elements of the group consisting of xanthan gum, acacia gum exudate, the mixture of xanthan gum (G_{X}) and of acacia gum exudate (G_{A}) in a mass ratio of xanthan gum (G_{X}) to acacia gum exudate (G_{A}) of greater than or equal to ⅓ and less than or equal to 3/1, the copolymer of the sodium salt of 2-acrylamido-2-methylpropanesulfonic acid and of 2-hydroxyethyl acrylate, crosslinked with triallylamine and/or with trimethylolpropane triacrylate and/or with methylenebis(acrylamide), the copolymer of the sodium salt of 2-acrylamido-2-methylpropanesulfonic acid and of acrylamide, crosslinked with triallylamine and/or with trimethylolpropane triacrylate and/or methylenebis(acrylamide), and the terpolymer, crosslinked with trimethylolpropane triacrylate and/or triallylamine and/or methylenebis(acrylamide), of 2-methyl-2-[(1-oxo-2-propenyl)amino]-1-propanesulfonic acid partially or completely salified in the sodium salt or ammonium salt form, in a molar proportion of greater than or equal to 60% and of less than or equal to 80%, of N,N-dimethylacrylamide, in a molar proportion of greater than or equal to 15% and of less than or equal to 39.5%, and tetraethoxylated lauryl methacrylate, in a molar proportion of greater than or equal to 0.5% and of less than or equal to 5%.

Composition (C₁) for topical use as defined previously may also comprise one or more auxiliary compounds chosen from solvents and cosolvents, or agents for improving skin penetration.

A subject of the present invention is also a composition (C′1) for topical use which is in the form of a gel and which comprises, per 100% of its mass:
from 50% to 80% by mass, preferably from 50% to 75% by mass and even more preferentially from 60% to 70% by mass of the lyzate (Ly) as defined previously,
from 0.1% to 5% by mass, preferably from 0.5% to 3% by mass, of at least one thickener and/or gelling agent, and
from 15% to 49.9% by mass, preferably from 22% to 49.5% by mass and even more preferentially from 27% to 39.5% by mass of at least one solvent.

Preferably, the solvent is chosen from the elements of the group consisting of:
the compounds of formula (Ia):

HO—[CH₂—CH(OH)—CH₂—O]ₙ—H (Ia)

in which n represents an integer greater than or equal to 1 and less than or equal to 15, more particularly greater than or equal to 1 and less than or equal to 10, more particularly greater than or equal to 1 and less than or equal to 6, more particularly greater than or equal to 1 and less than or equal to 4, more particularly equal to 1 or 2 or 3 or 4;
the compounds of formula (Ib):

Ra1-C(Rb1)(OH)—C(OH)(Rc1)(Rd1) (Ib),

in which each of the radicals Ra1, Rb1, Rc1 and Rd1 represent, independently of each other, a hydrogen atom or a saturated aliphatic radical including from 1 to 5 carbon atoms, or of the formula (Ib1):

Ra1-C(Rb1)(OH)—[C(Re1)(Rf1)]t-C(OH)(Rc1)(Rd1) (Ib1),

in which t is equal to 1, 2 or 3 and each of the radicals Ra1, Rb1, Rc1, Rd1, Re1 and Rf1 represent independently of each other a hydrogen atom or a saturated aliphatic radical including from 1 to 5 carbon atoms, it being understood that at least one of the radicals Ra1 or Rb1 and/or at least one of the radicals Rc1 or Rd1 does not represent a hydrogen atom.

Even more preferentially, the solvent will be glycerol.

This composition (C′1) may be administered topically to improve the moisturization of the skin and/or the scalp and/or the lips.

Compositions (C₁) and (C′₁) for topical use as defined previously may also comprise one or more cosolvents and agents for improving skin penetration.

As examples of cosolvents that may be present in compositions (C₁) and (C′₁) for topical use which are subjects of the present invention, mention may be made of water, organic solvents, for example glycerol, diglycerol, glycerol oligomers, ethylene glycol, propylene glycol, butylene glycol, hexylene glycol, diethylene glycol, xylitol, erythritol, sorbitol, water-soluble alcohols, such as ethanol, isopropanol or butanol, mixtures of water and of said organic solvents, propylene carbonate, ethyl acetate and benzyl alcohol.

As examples of agents for improving the skin penetration that may be present in compositions (C₁) and (C′₁) for topical use which are subjects of the present invention, mention may be made of glycol ethers, for instance ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether and diethylene glycol mono(n-butyl) ether, diethylene glycol monoethyl ether (or Transcutol-P), fatty acids, such as oleic acid, fatty acid esters of glycerol, for instance glyceryl behenate, glyceryl palmitate/stearate or behenoyl macroglycerides, polyoxyethylene (2) stearyl ether, polyoxyethylene (2) oleyl ether, terpenes, for instance D-limonene, or essential oils, for instance eucalyptus essential oil.

A subject of the present invention is also a composition (C2) for topical use which is in the form of an emulsion of water-in-oil type or of an emulsion of oil-in-water type, comprising the lyzate (Ly) as defined previously, and preferably obtained by performing a process including steps a) to f) as described above.

A distinction is made between oil-in-water (O/W) emulsions in which the continuous phase consists of a hydrophilic phase, generally an aqueous phase, and the dispersed phase consists of a lipophilic fatty phase, and water-in-oil (W/O) emulsions in which the continuous phase consists of a lipophilic fatty phase and the dispersed phase consists of a hydrophilic phase, generally an aqueous phase.

This composition (C2) may also be administered topically for improving the moisturization of the skin and/or the scalp and/or the lips.

Composition (C2) may in particular be administered topically for reducing and/or eliminating and/or preventing chapping and/or dry patches and/or cracks and/or atopic dermatitis and/or ichthyosis and/or dryness of the skin or mucous membranes accompanying cutaneous and/or mucosal pathologies such as eczema.

Among the emulsions of water-in-oil type, a particular subject of the present invention is a composition (F) for topical use which is in the form of a water-in-oil emulsion and which comprises, per 100% of its mass:
from 60% to 90% by mass of composition (C1) or (C′1) for topical use as defined in one of embodiments 3 to 9,
from 10% to 40% by mass of a fatty phase (A₂) comprising i) at least one oil, and optionally at least one wax, and ii) an emulsifying system comprising at least one emulsifying surfactant (Si).

Preferably, the emulsifying surfactant (Si) will be chosen from the elements of the group consisting of alkylpolyglycoside compositions, compositions of alkylpolyglycosides and of fatty alcohols, polyglycerol esters, alkoxylated polyglycerol esters, polyglycol polyhydroxystearates, polyglycerol polyhydroxystearates and alkoxylated polyglycerol polyhydroxystearates.

In formulation (F) for topical use which is a subject of the present invention, the term “oil” denotes a water-insoluble compound and/or mixture of compounds, which is liquid at 25° C., and more particularly:
linear alkanes including from 11 to 19 carbon atoms;
branched alkanes including from 7 to 40 carbon atoms, such as isododecane, isopentadecane, isohexadecane, isoheptadecane, isooctadecane, isononadecane or isoeicosane, or mixtures of some of them such as those mentioned below and identified by their INCI name: C₇₋₈ isoparaffin, C₈₋₉ isoparaffin, C₉-11 isoparaffin, C₉₋₁₂ isoparaffin, C₉₋₁₃ isoparaffin, C₉₋₁₄ isoparaffin, C₉₋₁₆ isoparaffin, C₁₀₋₁₁ isoparaffin, C₁₀₋₁₂ isoparaffin, C₁₀₋₁₃ isoparaffin, C₁₁₋₁₂ isoparaffin, C₁₁₋₁₃ isoparaffin, C₁₁₋₁₄ isoparaffin, C₁₂₋₁₄ isoparaffin, C₁-20 isoparaffin, C₁₃₋₄ isoparaffin, C₁₃₋₆ isoparaffin;
cycloalkanes optionally substituted with one or more linear or branched alkyl radicals;
white mineral oils, such as the products sold under the following names: Marcol™ 52, Marcol™ 82, Drakeol™ 6VR, Eolane™ 130, Eolane™ 150;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
mixtures of alkanes including from 15 to 19 carbon atoms, said alkanes being linear alkanes, branched alkanes and cycloalkanes, and more particularly the mixture (M₁) which comprises, per 100% of its mass, a mass proportion of branched alkanes of greater than or equal to 90% and less than or equal to 100%; a mass proportion of linear alkanes of greater than or equal to 0% and less than or equal to 9%, and more particularly less than 5%, and a mass proportion of cycloalkanes of greater than or equal to 0% and less than or equal to 1%, for example the mixtures sold under the name Emogreen™ L15 or Emogreen™ L19;
the fatty alcohol ethers of formula (II):

Z₁—O—Z₂ (II),

in which Z₁ and Z₂, which may be identical or different, represent a linear or branched alkyl radical including from 5 to 18 carbon atoms, for example dioctyl ether, didecyl ether, didodecyl ether, dodecyl octyl ether, dihexadecyl ether, (1,3-dimethylbutyl) tetradecyl ether, (1,3-dimethylbutyl) hexadecyl ether, bis(1,3-dimethylbutyl) ether or dihexyl ether;
monoesters of fatty acids and of alcohols of formula (III):

R′₁—(C═O)—O—R′₂ (III),

in which R′₁—(C═O) represents a saturated or unsaturated, linear or branched acyl radical including from 8 to 24 carbon atoms, and R′2 represents, independently of R′₁, a saturated or unsaturated, linear or branched hydrocarbon-based chain including from 1 to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, isostearyl isostearate;
diesters of fatty acids and of glycerol of formula (IV) and of formula (V):

R′₃—(C═O)—O—CH₂—CH(OH)—CH₂—O—(C═O)—R′₄ (IV)

R′₅—(C═O)—O—CH₂—CH[O—(C═O)—R′₆]—CH₂—OH (V),

in which formulae (VI) and (VII) R′₃—(C═O), R′₄—(C═O), R'₅—(C═O) and R′₆—(C═O), which may be identical or different, represent a saturated or unsaturated, linear or branched acyl group including from 8 to 24 carbon atoms;
triesters of fatty acids and of glycerol of formula (VI):

R′₇—(C═O)—O—CH₂—CH[O—(C═O)—R″₈]—CH₂—O—(C═O)—R″₉ (VI),

in which R′₇—(C═O), R'₈—(C═O) and R′₉—(C═O), which may be identical or different, represent a saturated or unsaturated, linear or branched acyl group including from 8 to 24 carbon atoms;
plant oils, such as phytosqualane, sweet almond oil, coconut kernel oil, castor oil, jojoba oil, olive oil, rapeseed oil, groundnut oil, sunflower oil, wheat germ oil, corn germ oil, soybean oil, cottonseed oil, alfalfa oil, poppy seed oil, pumpkin seed oil, evening primrose oil, millet oil, barley oil, rye oil, safflower oil, candlenut oil, passionflower oil, hazelnut oil, palm oil, shea butter, apricot kernel oil, beauty-leaf oil, sisymbrium oil, avocado oil, calendula oil, oils derived from flowers or vegetables;
ethoxylated plant oils.

Preferably, the composition according to the invention comprises at least one oil chosen from the elements of the group consisting of castor oil, liquid paraffins, cocoyl caprate/caprylate, isopropyl myristate and capric/caprylic triglyceride.

The fatty phase (A2) optionally comprises wax. In formulation (F) for topical use which is a subject of the present invention, the term “wax” denotes a water-insoluble compound and/or mixture of compounds, which is solid at 35° C.

Such a wax is more particularly chosen from beeswax, carnauba wax, candelilla wax, ouricury wax, Japan wax, cork fibre wax, sugarcane wax, paraffin waxes, lignite waxes, microcrystalline waxes, lanolin wax; ozokerite; polyethylene wax; silicone waxes; plant waxes; fatty alcohols and fatty acids that are solid at room temperature; glycerides that are solid at room temperature.

In the definition of formulation (F) which is a subject of the present invention, the term “alkylpolyglycoside composition” denotes a composition (A) represented by formula (VII):

R₁—O-(G)₁-H (VII)

in which x represents a decimal number between 1.05 and 5, G represents a reducing sugar residue, and R₁ represents a saturated or unsaturated, linear or branched aliphatic hydrocarbon-based radical, optionally substituted with one or more hydroxyl groups, including from 12 to 36 carbon atoms, said composition (A) consisting of a mixture of compounds represented by formulae (VII₁), (VII₂), (VII₃), (VII₄) and (VII₅):

R₁—O-(G)₁-H (VII₁)

R₁—O-(G)₂-H (VII₂)

R₁—O-(G)₃-H (VII₃)

R₁—O-(G)₄-H (VII₄)

R₁—O-(G)₅-H (VII₅)

in the respective molar proportions a₁, a₂, a₃, a₄ and as such that:
the sum a₁+a₂+a₃+a₄+a₅ is equal to 1 and that
the sum a₁+2a₂+3a₃+4a₄+5a₅ is equal to x.

The term “saturated or unsaturated, linear or branched aliphatic hydrocarbon-based radical including from 12 to 36 carbon atoms, optionally substituted with one or more hydroxyl groups” denotes, for the radical R₁ in formula (VII) as defined above:
saturated linear alkyl radicals, for example n-dodecyl, n-tridecyl, n-tetradecyl, n-pentadecyl, n-hexadecyl, n-heptadecyl, n-octadecyl, n-nonadecyl, n-eicosyl and n-docosyl radicals;
unsaturated linear radicals such as dodecenyl, tridecenyl, tetradecenyl, pentadecenyl, hexadecenyl, heptadecenyl, octadecenyl, nonadecenyl, eicosenyl, docosenyl, 4-dodecenyl and 5-dodecenyl radicals;
saturated or unsaturated, linear or branched aliphatic radicals including from 12 to 36 carbon atoms substituted with one or two hydroxyl groups, such as hydroxydodecyl, hydroxytetradecyl, hydroxyhexadecyl, hydroxyoctadecyl, hydroxyeicosyl and hydroxydocosyl radicals, for example the 12-hydroxyoctadecyl radical;
radicals derived from the isoalkanols of formula (1):

(CH₃)(CH₃)CH—(CH₂)ᵣ—CH₂—OH (1)

in which r represents an integer between 8 and 20, for example the isodecyl, isoundecyl, isododecyl, isotridecyl, isotetradecyl, isopentadecyl, isohexadecyl, isopentadecyl, isooctadecyl, isononadecyl, isoeicosyl or isodocosyl radicals;
branched alkyl radicals, derived from Guerbet alcohols, of formula (2):

CH(CₛH₂ₛ₊₁)(CₜH₂ₜ₊₁)—CH₂—OH (2)

in which t is an integer between 6 and 18, s is an integer between 4 and 18 and the sum s+t is greater than or equal to 10 and less than or equal to 22, for example the 2-butyloctyl, 2-butyldecyl, 2-hexyloctyl, 2-hexyldecyl, 2-octyldecyl, 2-hexyldodecyl, 2-octyldodecyl, 2-decyltetradecyl, 2-dodecylhexadecyl or 2-tetradecyloctadecyl radicals.

According to a particular aspect, in the definition of formula (VII) as defined above, R₁ represents a saturated or unsaturated, linear or branched aliphatic hydrocarbon-based radical including from 12 to 24 carbon atoms.

The term “reducing sugar” in the definition of formula (VII) as defined above denotes saccharide derivatives that do not have in their structures any glycoside bonds established between an anomeric carbon and the oxygen of an acetal group as defined in the reference publication: “Biochemistry”, Daniel Voet/Judith G. Voet, page 250, John Wiley & Sons, 1990. The oligomeric structure (G)ₓ may exist in any isomeric form, whether it is optical isomerism, geometrical isomerism or regioisomerism; it may also represent a mixture of isomers.

In formula (VII) as defined above, the group R₁—O— is linked to G via the anomeric carbon of the saccharide residue, so as to form an acetal function.

According to a particular aspect in the definition of formula (VII) as defined above, G represents a reducing sugar residue chosen from glucose, dextrose, sucrose, fructose, idose, gulose, galactose, maltose, isomaltose, maltotriose, lactose, cellobiose, mannose, ribose, xylose, arabinose, lyxose, allose, altrose, dextran and tallose; and more particularly, G represents a reducing sugar residue chosen from glucose, xylose and arabinose residues.

According to an even more particular aspect, in the definition of formula (VII), x represents a decimal number greater than or equal to 1.05 and less than or equal to 2.5, more particularly greater than or equal to 1.05 and less than or equal to 2.0 and even more particularly greater than or equal to 1.25 and less than or equal to 2.0.

According to an even more particular aspect, in the definition of formula (VII) as defined above, R₁ represents the radical chosen from at least one of the elements of the group consisting of n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl and 2-decyltetradecyl radicals; G represents a reducing sugar residue chosen from glucose and xylose residues and x represents a decimal number greater than or equal to 1.05 and less than or equal to 2.5.

According to an even more particular aspect, formulation (F) as defined previously is characterized in that said emulsifying system consists of an alkylpolyglycoside composition (A) represented by formula (VII):

R₁—O-(G)ₓ-H (VII)

in which x represents a decimal number between 1.05 and 2.5, G represents a xylose residue, and R₁ represents a 2-octyldodecyl radical, said composition (C₂) consisting of a mixture of compounds represented by formulae (VII₁), (VII₂), (VII₃), (VII₄) and (VII₅):

R₁—O-(G)₁-H (VII₁)

R₁—O-(G)₂-H (VII₂)

R₁—O-(G)₃-H (VII₃)

R₁—O-(G)₄-H (VII₄)

R₁—O-(G)₅-H (VII₅)

in the respective molar proportions a₁, a₂, a₃, a₄ and as such that:
the sum a₁+a₂+a₃+a₄+a₅ is equal to 1 and that
the sum a₁+2a₂+3a₃+4a₄+5a₅ is equal to x.

In the definition of composition (F) which is a subject of the present invention, the term “alkylpolyglycoside and fatty alcohol composition” denotes a composition (B) comprising, per 100% of its mass:
from 10% to 50% by mass, more particularly from 15% to 40% by mass and even more particularly from 20% to 30% by mass of at least one composition (A) represented by formula (VII) as defined previously,
from 90% to 50% by mass, more particularly from 85% to 60% by mass and even more particularly from 80% to 70% by mass of at least one fatty alcohol of formula (VIII):

R′₁—OH (VIII),

in which R′₁, which may be identical to or different from R₁, represents a saturated or unsaturated, linear or branched aliphatic hydrocarbon-based radical, optionally substituted with one or more hydroxyl groups, including from 12 to 36 carbon atoms.

According to a particular aspect, in the definition of formula (VII) representing composition (A) included in composition (B), R₁ represents the radical chosen from n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl and 2-decyltetradecyl radicals, G represents a reducing sugar residue chosen from glucose and xylose residues and x represents a decimal number greater than or equal to 1.05 and less than or equal to 2.5.

According to a more particular aspect, in the definition of formula (VII) representing composition (A) included in composition (B), R₁ represents a 2-octyldodecyl radical, G represents a xylose residue and x represents a decimal number greater than or equal to 1.05 and less than or equal to 2.5.

According to a more particular aspect, in the definition of the fatty alcohol of formula (VIII) as defined above, R′₁ represents a radical chosen from n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl and 2-decyltetradecyl radicals, R′₁ most particularly represents a 2-octyldodecyl radical.

According to an even more particular aspect, a subject of the invention is a formulation (F) as defined previously, characterized in that said emulsifying system consists of a composition (B) comprising, per 100% of its mass:
from 10% to 50% by mass of at least one alkylpolyglycoside composition (A) represented by formula (VII):

R₁—O-(G)ₓ-H (VII)

in which x represents a decimal number between 1.05 and 2.5, G represents a xylose residue, and R₁ represents a 2-octyldodecyl radical, said composition consisting of a mixture of compounds represented by formulae (VII₁), (VII₂), (VII₃), (VII₄) and (VII₅):

R₁—O-(G)₁-H (VII₁)

R₁—O-(G)₂-H (VII₂)

R₁—O-(G)₃-H (VII₃)

R₁—O-(G)₄-H (VII₄)

R₁—O-(G)₅-H (VII₅)

in the respective molar proportions a₁, a₂, a₃, a₄ and as such that:
the sum a₁+a₂+a₃+a₄+a₅ is equal to 1 and that
the sum a₁+2a₂+3a₃+4a₄+5a₅ is equal to x; and
from 90% to 50% by mass of at least one fatty alcohol of formula (VIII):

R′₁—OH (VIII),

in which R′₁ represents a 2-octyldodecyl radical.

In the definition of formulation (F) which is a subject of the present invention, the term “polyglycerol ester” denotes a compound of formula (IX):

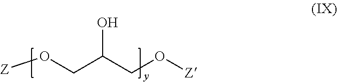

in which Z represents an acyl radical of formula R₂—C(═O)—, in which R₂ represents a saturated or unsaturated, linear or branched aliphatic hydrocarbon-based radical, including from 11 to 35 carbon atoms and more particularly a radical chosen from dodecanoyl, tetradecanoyl, hexadecanoyl, octadecanoyl, eicosanoyl, docosanoyl, oleyl, linoleyl, linolenoyl and isostearyl radicals, Z′ represents the acyl radical of formula R₂—C(═O)— as defined above, with Z′ being identical to or different from Z, or a hydrogen atom, and y represents an integer greater than than or equal to 2 and less than or equal to 20.

According to a more particular aspect, the compound of formula (IX) is chosen from the elements of the group consisting of decaglyceryl oleate, decaglyceryl isostearate, decaglyceryl monolaurate, decaglyceryl monolinoleate and decaglyceryl monomyristate.

In the definition of formulation (F) which is a subject of the present invention, the term “alkoxylated polyglycerol ester” denotes a compound of formula (X):

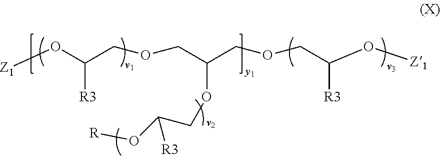

in which Z₁ represents an acyl radical of formula R′₂—C(═O)—, in which R′₂ represents a saturated or unsaturated, linear or branched aliphatic hydrocarbon-based radical, including from 11 to 35 carbon atoms, and more particularly a radical chosen from dodecanoyl, tetradecanoyl, hexadecanoyl, octadecanoyl, eicosanoyl, docosanoyl, oleyl, linoleyl, linolenoyl and isostearyl radicals, Z₁′ represents the acyl radical of formula R′₂—C(═O)— as defined above, with Z₁′, which may be identical to or different from Z₁, or a hydrogen atom, R₃ represents a hydrogen atom, a methyl radical or an ethyl radical, y₁ represents an integer greater than than or equal to 2 and less than or equal to 20, v₁, v₂ and v₃, which may be identical or different, represent an integer greater than or equal to 0 and less than or equal to 50, and the sum [(y₁. v₁)+(y₁. v₂)+v₃)] is an integer greater than or equal to 1 and less than or equal to 50.

In the definition of composition (F) which is a subject of the present invention, the term “polyglycol polyhydroxystearate” denotes a compound of formula (XI):

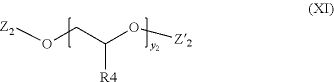

in which y₂ represents an integer greater than or equal to 2 and less than or equal to 50, R₄ represents a hydrogen atom, a methyl radical or an ethyl radical, and Z₂ represents a radical of formula (XII):

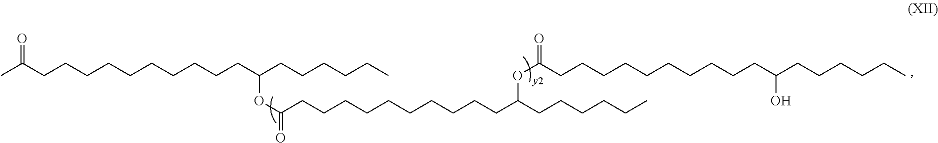

in which y′₂ represents an integer greater than or equal to 0 and less than or equal to 10, more particularly greater than or equal to 1 and less than or equal to 10 and Z′₂ represents a radical of formula (XII) as defined above, with Z₂′ which may be identical to or different from Z₂, or a hydrogen atom.

In the definition of formulation (F) which is a subject of the present invention, the term “polyglycerol polyhydroxystearate” denotes a compound of formula (XIII):

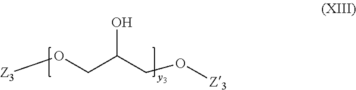

in which Z₃ represents a radical of formula (XII) as defined above, Z′₃ represents a radical of formula (XII) as defined above, with Z₃′ which may be identical to or different from Z₃, or a hydrogen atom, y₃ represents an integer greater than or equal to 2 and less than or equal to 20.

In the definition of formulation (F) which is a subject of the present invention, the term “alkoxylated polyglycerol polyhydroxystearate” denotes a compound of formula (XIV):

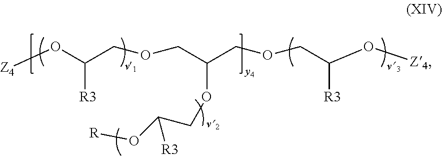

in which Z₄ represents a radical of formula (XII) as defined above, Z′₄ represents a radical of formula (XII) as defined above, with Z₄′ being identical to or different from Z₄, or a hydrogen atom, y₄ represents an integer greater than or equal to 2 and less than or equal to 20, v′₁, v′₂ and v′₃, which may be identical or different, represent an integer greater than or equal to 0 and less than or equal to 50, and the sum [(y₄. v′₁)+(y₄. v′₂)+v′₃)] is an integer greater than or equal to 1 and less than or equal to 50.

According to another particular aspect, a subject of the invention is a formulation (F) as defined previously, characterized in that said emulsifying system (S) consists of a composition (D) comprising, per 100% of its mass:
from 15% to 25% by mass of at least one composition (A) represented by formula (VII):

R1-O-(G)ₓ-H (VII)

in which x represents a decimal number between 1.05 and 2.5, G represents a xylose residue, and R1 represents a 2-octyldodecyl radical, said composition (A) consisting of a mixture of compounds represented by formulae (VII₁), (VII₂), (VII₃), (VII₄) and (VII₅):

R₁—O-(G)₁-H (VII₁)

R₁—O-(G)₂-H (VII₂)

R₁—O-(G)₃-H (VII₃)

R₁—O-(G)₄-H (VII₄)

R₁—O-(G)₅-H (VII₅)

in the respective molar proportions a₁, a₂, a₃, a₄ and as such that:
the sum a₁+a₂+a₃+a₄+a₅ is equal to 1 and that
the sum a₁+2a₂+3a₃+4a₄+5a₅ is equal to x;
from 55% to 65% by mass of at least one fatty alcohol of formula (VIII):

R′₁—OH (VIII),

in which R′₁ represents a 2-octyldodecyl radical;
from 10% to 30% by mass of at least one polyglycol polyhydroxystearate represented by formula (XI):

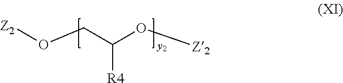

in which y₂ represents an integer greater than or equal to 2 and less than or equal to 50, R₄ represents a hydrogen atom, a methyl radical or an ethyl radical, and Z₂ represents a radical of formula (XII):

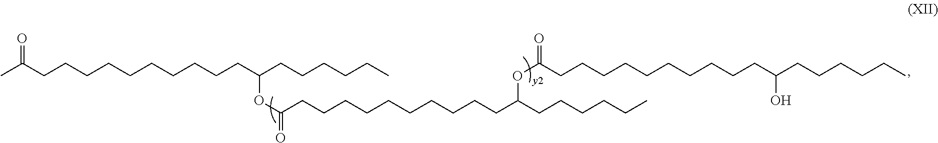

in which y′₂ represents an integer greater than or equal to 0 and less than or equal to 10, more particularly greater than or equal to 1 and less than or equal to 10, and Z′₂ represents a radical of formula (XII) as defined above, with Z₂′ being identical to or different from Z₂, or a hydrogen atom.

According to another particular aspect, a subject of the invention is a formulation (F) as defined previously, characterized in that the dynamic viscosity of said formulation (F), measured at a temperature of 25° C. using a Brookfield LVT viscometer at a speed of 6 rpm, is greater than or equal to 500 mPa·s and less than or equal to 40 000 mPa·s.

A subject of the present invention is also a composition (F′) for topical use which is in the form of an oil-in-water emulsion and which comprises, per 100% of its mass:
from 50% to 90% by mass, preferably from 60% to 90% by mass and even more preferentially from 70% to 90% by mass of a cosmetically acceptable aqueous phase (A1), said aqueous phase (A1) comprising, per 100% of its own mass, from 0.5% to 10% by mass of the lyzate (Ly) as defined previously,
from 10% to 50% by mass, preferably from 10% to 40% by mass and even more preferentially from 10% to 30% by mass of a fatty phase (G1) comprising, per 100% of its own mass:
   from 0.5% to 20% by mass, preferably from 1% to 15% by mass, of at least one surfactant of oil-in-water type (S′1),
   from 80% to 99.5% by mass of at least one oil and/or one wax.

For the purposes of the present invention, the term “oil” present in the fatty phase (G₁) of composition (F′) which is in the form of an emulsion of oil-in-water type as defined previously denotes chemical substances or mixtures of chemical substances which are water-insoluble and which have a liquid appearance at a temperature of 25° C.

For the purposes of the present invention, the term “wax” present in the fatty phase (G₁) of composition (F′) which is in the form of an emulsion of oil-in-water type as defined previously denotes chemical substances or mixtures of chemical substances which are water-insoluble and which have a solid appearance at a temperature of 45° C.

For the purposes of the present invention, the term “surfactant of oil-in-water type (S′1)” present in the fatty phase (G₁) of composition (F′) which is in the form of an emulsion of oil-in-water type as defined previously denotes the chemical substance or the mixture of chemical substances which makes it possible to stabilize the droplets of said fatty phase (G₁) in dispersion in the continuous aqueous phase (A₁).

As surfactant of oil-in-water type (S′1) present in the fatty phase (G1) of the emulsion (F′) of oil-in-water type as defined previously, mention may be made, for example, of:
polysorbates resulting from the ethoxylation reaction between one molar equivalent of sorbitan esters and between 5 and 20 molar equivalents of ethylene oxide, and more particularly between one molar equivalent of sorbitan laurate or of sorbitan palmitate or of sorbitan stearate or of sorbitan isostearate or of sorbitan oleate and between 5 and 20 molar equivalents of ethylene oxide, more particularly 5 or 10 or 12 or 15 or 20 molar equivalents of ethylene oxide;
the products resulting from the ethoxylation reaction between one molar equivalent of a fatty acid, for instance palmitic acid, myristic acid, lauric acid, stearic acid, isostearic acid or oleic acid, and between 5 and 40 molar equivalents of ethylene oxide;
the products resulting from the esterification reaction between a fatty acid, for instance palmitic acid, myristic acid, lauric acid, stearic acid, isostearic acid, oleic acid, arachidic acid or behenic acid and between 4 to 20 molar equivalents, more particularly between 3 to 10 molar equivalents of glycerol;

For the purposes of the present invention, the term “fatty phase (G1)” denotes a fatty substance or a mixture of fatty substances which is insoluble in water and/or in mixtures of water and of polar solvents. Such a “fatty phase” may comprise oils and/or waxes as defined previously.

Among the constituent elements of the fatty phase (G1) present in formulation (F′), mention may be made of the constituent oils of the fatty phase (A2) as described above for the preparation of formulation (F) and of silicone oils, such as dimethylpolysiloxanes, methylphenylpolysiloxanes, silicones modified with amines, silicones modified with fatty acids, silicones modified with alcohols, silicones modified with alcohols and fatty acids, silicones modified with polyether groups, modified epoxy silicones, silicones modified with fluorinated groups, cyclic silicones and silicones modified with alkyl groups.

Among the constituent elements of the fatty phase (G1) present in formulation (F′), mention may be made of the constituent waxes of the fatty phase (A2) such as described above for the preparation of formulation (F).

According to a particular aspect, in formulation (F′) which is a subject of the present invention, the fatty phase (G1) also comprises, per 100% of its own mass, from 5% to 30% by mass, more particularly from 5% to 25% by mass and even more particularly from 10% to 25% by mass of at least one agent for protecting against the ultraviolet rays of the sun.

The term “agent for protecting against the ultraviolet rays of the sun” notably denotes, in the definition of composition (F′) for topical use which is in the form of an emulsion of oil-in-water type and which is a subject of the present invention, pigments, organic sunscreens and mineral sunscreens.

As pigments used as agents for protecting against the ultraviolet rays of the sun, there are, for example, titanium dioxide, brown iron oxides, yellow iron oxides, black iron oxides or red iron oxides, or else white or colored nacreous pigments such as titanium mica.

As organic sunscreens used as agents for protecting against the ultraviolet rays of the sun, there are, for example:
those of the family of benzoic acid derivatives, such as para-aminobenzoic acids (PABAs), notably monoglyceryl esters of PABA, ethyl esters of N,N-propoxy PABA, ethyl esters of N,N-diethoxy PABA, ethyl esters of N,N-dimethyl PABA, methyl esters of N,N-dimethyl PABA, butyl esters of N,N-dimethyl PABA;
those of the family of anthranilic acid derivatives, such as homomenthyl-N-acetyl anthranilate;
those of the family of salicylic acid derivatives, such as amyl salicylate, homomenthyl salicylate, ethylhexyl salicylate, phenyl salicylate, benzyl salicylate, p-isopropanolphenyl salicylate;
those of the family of cinnamic acid derivatives, such as ethylhexyl cinnamate, ethyl 4-isopropylcinnamate, methyl 2,5-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxycinnamate, ethyl α-cyano-p-phenylcinnamate, 2-ethylhexyl α-cyano-p-phenylcinnamate or mono(2-ethylhexanoyl)glyceryl di(para-methoxycinnamate);
those of the family of benzophenone derivatives, such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxy benzophenone-5-sulfonate, 4-phenylbenzophenone, 2-ethylhexyl-4′-phenyl benzophenone-2-carboxylate, 2-hydroxy-4-n-octyloxybenzophenone, 4-hydroxy-3-carboxybenzophenone; 3-(4′-methylbenzylidene)-D,L-camphor, 3-(benzylidene)-D,L-camphor, camphor benzalkonium methosulfate; urocanic acid, ethyl urocanate;
those of the family of sulfonic acid derivatives, such as 2-phenylbenzimidazole-5-sulfonic acid and salts thereof; the family of triazine derivatives, such as hydroxyphenyltriazine, ethylhexyloxyhydroxyphenyl-4-methoxyphenyltriazine, benzoic acid 2,4,6-trianillino-(p-carbo-2′-ethylhexyl-1′-oxy)-1,3,5-triazine, 4,4-((6-(((1,1-dimethylethyl)amino)carbonyl)phenyl)amino)-1,3,5-triazine-2,4-diyldiimino)bis(2-ethylhexyl) ester, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; 2-(4-diethylamino-2-hydroxybenzoyl)benzoic acid hexyl ester, 2,4-bis{[4-(2-ethylhexyloxy)-2-hydroxy]phenyl}-6-(4-methoxyphenyl)-1,3,5-triazine, 2,4,6-tris[4-(2-ethylhexyloxycarbonyl)anilino]-1,3,5-triazine, 2-ethylhexyl dimethoxybenzylidene dioxoimidazolidine propionate, the family of diphenylacrylate derivatives, such as 2-ethylhexyl 2-cyano-3,3-diphenyl-2-propenoate, ethyl 2-cyano-3,3-diphenyl-2-propenoate;
those of the family of polysiloxanes, such as benzylidene siloxane malonate.

As inorganic sunscreens used as agents for protecting against the ultraviolet rays of the sun, there are, for example, titanium oxides, zinc oxides, cerium oxide, zirconium oxide, yellow, red or black iron oxides, and chromium oxides. These mineral sunblocks may or may not be micronized, may or may not have been subjected to surface treatments and may optionally be in the form of aqueous or oily predispersions.

The agent for protecting against the ultraviolet rays of the sun will preferably be chosen from the elements of the group consisting of titanium dioxide, 2,4-dihydroxybenzophenone, 2-(4-diethylamino-2-hydroxybenzoyl)benzoic acid hexyl ester, 2,4-bis[4-(2-ethylhexyloxy)-2-hydroxyphenyl]-6-(4-methoxyphenyl)-1,3,5-triazine, 2,4,6-tris[4-(2-ethylhexyloxycarbonyl)anilino]-1,3,5-triazine and 2-ethylhexyl dimethoxybenzylidene dioxoimidazolidine propionate.

The term “for topical use” used in the definition of composition (E1) in the form of an emulsion of oil-in-water type as defined above means that said composition is formulated to allow its application to the skin, the hair, the scalp or the mucous membranes, whether it is a direct application in the case of a cosmetic, dermocosmetic, dermopharmaceutical or pharmaceutical composition or an indirect application, for example in the case of a body hygiene product in the form of a textile or paper wipe, or sanitary products intended to be in contact with the skin or the mucous membranes.

The term “cosmetically acceptable” used in the definition of the aqueous phase (A₁) of composition (E1) in the form of an emulsion of oil-in-water type means, according to the Council of the European Economic Community Directive No. 76/768/EEC of Jul. 27, 1976, amended by Directive No. 93/35/EEC of Jun. 14, 1993, any substance or preparation intended to be brought into contact with the various parts of the human body (epidermis, bodily hair and head hair system, nails, lips and genitals) or with the teeth and oral mucosae, for the purpose, exclusively and mainly, of cleansing them, fragrancing them, modifying the appearance thereof and/or correcting bodily odors thereof and/or protecting them or keeping them in good condition.

Compositions (F) and (F′) may be administered topically for improving the moisturization of the skin and/or the scalp and/or the lips.

Compositions (F) and (F′) may in particular be administered topically for reducing and/or eliminating and/or preventing chapping and/or dry patches and/or cracks and/or atopic dermatitis and/or ichthyosis and/or dryness of the skin or mucous membranes accompanying cutaneous and/or mucosal pathologies such as eczema.

### DESCRIPTION OF THE PREFERRED EMBODIMENTS

### 1) Preparation of a Lyzate According to the Invention (Ly)

A lyzate (Ly) of dedifferentiated cells of the plant Helichrysum stoechas is prepared by performing the process described previously and more particularly by performing:
a step a) of preparing the sterile Helichrysum stoechas sample, as described previously, using Helichrysum stoechas leaves cut into fragments of a few millimeters, and then sterilized by successive passage:
in a bath of 70% ethanol and Tween™ 80 (at 0.05%) for five minutes
in a 1% aqueous bleach bath for five minutes.

The leaves are subsequently rinsed in sterile distilled water three times in succession.
A step b) of callogenesis, as described previously, with a callogenesis medium, the constitution of which is described in table 1 above, with an incubation phase having a duration of three weeks at a temperature of 20 to 25° C. in the light.
A step c) of suspending the dedifferentiated cells, as described previously, with a culture medium, the composition of which is described in table 2 above, for a period of 14 to 30 days, at a temperature of 20 to 25° C., in daylight, agitated by orbital-type agitation at a speed of 100 rpm.
A step d) of selection of a fine suspension as described previously, with withdrawal of ⅙ to ¼ of the culture obtained in step c), to which is added a volume of suspension medium as described in table 2 and in a volume adjusted to the volume of inoculum available in the container.
The new suspension is cultured in a climatically controlled chamber for a period of 14 days, at a temperature of 20 to 25° C., in daylight, agitated by orbital-type agitation at a speed of 100 rpm.
A step e) of production of biomass as described previously, with withdrawal of ⅙ to ¼ of the medium obtained in step d), to which is added a volume of suspension medium as described in table 2 and in a volume adjusted to the volume of inoculum available in the container.
The new suspension is cultured in a climatic chamber for a period of 14 days in a wave bioreactor, with a capacity of between 5 liters and 1000 liters, at a temperature of 20 to 25° C., in daylight, agitated by orbital-type agitation at a speed of 25 rpm.
A step f) of high pressure homogenization of the medium obtained on conclusion of step e), by passing it through a GEA brand high pressure homogenizer, at a pressure of 500 bar on a pass at a temperature below 40° C.
A lyzate (Ly) is thus obtained comprising, per 100% of its mass:
90-98% by mass of water
10-2% by mass of solids
A step g) of stabilization of the medium obtained in step f), by mixing 60% by mass of the medium obtained in step f) with 0.8% by mass of a thickener sold under the brand name Solagum™ AX, 37.9% by mass of glycerol, 0.3% of potassium sorbate and 1% of sodium benzoate, brought to a pH value of 5.3.

A composition C_{A} is thus obtained comprising, per 100% of its mass:
60% by mass of lyzate (Ly)
37.9% by mass of glycerol
0.8% by mass of Solagum™ AX
0.3% by mass of potassium sorbate
1% by mass of sodium benzoate.
   2) Experimental Use of the Biological Effects of a Lyzate of Dedifferentiated Cells of the Plant Helichrysum stoechas According to the Invention

As already mentioned, the lyzate as defined previously and obtained by performing a process comprising steps a) to f) as described above, makes it possible to maintain a good level of moisturization and to contribute toward the comfort of dry, reactive and/or sensitive skin.

The particular technical effect of the lyzate (Ly) as defined previously and obtained by performing a process comprising steps a) to f) as described above, was demonstrated on several in vitro and in vivo study models.

The experiments were replicated twice.

The values were expressed as mean±sd [standard deviation].

For each condition, the percentages of stimulation or of protection were calculated as follows:

% restoration=100×[mean(composition C_{A})−mean(impaired epidermides)]/[mean(healthy epidermides)−mean(impaired epidermides)].

A—Moisturizing Effect

Tests were then performed to show the moisturizing effect of the lyzate (Ly) as defined previously.

Various analyses were performed to study the restoration of the barrier function following impairment by the “tape-stripping” method.

This method consists in applying a piece of adhesive tape (“D-Squame” brand) to the surface of the skin, pressing it on the surface of the skin and then removing it. The first applications remove a complete cell layer of corneocytes. The amount of corneocytes on the adhesive tape decreases as the applications progress.

The “D-Squame” adhesive tapes are applied to the explants, care being taken that they are always applied at the same place and in the same way, with a constant thumb pressure. The adhesive tape is subsequently removed in a smooth, rapid and unidirectional movement so as to obtain homogeneous removal of the corneocytes.

This model makes it possible to evaluate the impact of cosmetic formulations on the physiology of the epidermides, the barrier function of which is impaired.

According to the type of evaluation of the effect of improving the moisturization of the skin and/or scalp and/or lips, a formulation (F₁) comprising 1% of composition C_{A} is used and its mass constitution is as follows.

TABLE 3
Formulation (F₁)

| | Content as a |
|---|---|
| Ingredients | mass percentage |
| Water | QSP 100% |
| Glycerol | 1% |
| Montanov ™ L ⁽¹⁾ | 1 |
| Simulsol ™ 165⁽²⁾ | 1 |
| Triglycerides C8-C10 | 3 |
| Caprylic/Capric Triglycerides | |
| DUB ™ DNPG⁽³⁾ | 7 |
| Neopentyl glycol diheptanoate | |
| Sepimax ™ ZEN ⁽⁴⁾ | 1 |
| Polyacrylate Crosspolymer-6 | |
| Sepinov ™ EMT 10 ⁽⁵⁾ | 0.5 |
| Hydroxyethyl Acrylate/Sodium | |
| Acryloyldimethyl Taurate Copolymer | |
| Euxyl ™ PE 9010 ⁽⁶⁾ | 1 |
| Phenoxylethanol - Ethylhexylglycerin | |
| Sensiva ™ PA40 ⁽⁷⁾ | 0.5 |
| 3-Phenylpropan-1-ol-Octane-1,2-diol - | |
| Propane-1,3-diol | |
| Composition C_{A} | 1 |

⁽¹⁾ Montanov ™ L (INCI name: C14-22 Alcohols & C12-20 Alkyl Glucoside) is an emulsifying composition, intended to stabilize emulsions, and used for preparing cosmetic formulations.
⁽²⁾Simulsol ™ 165 (INCI name: PEG-100 Stearate and Glyceryl Stearate) is an emulsifying composition, intended for stabilizing emulsions, notably oil-in-water emulsions, and used for preparing cosmetic formulations.
⁽³⁾DUB ™ DNPG (INCI name: Neopentyl Glycol Diheptanoate) is an ester used as a fatty phase and/or emollient agent for preparing cosmetic formulations.
⁽⁴⁾ Sepimax ™ Zen (INCI name: Polyacrylate Crosspolymer-6) is a crosslinked anionic polyelectrolyte used as thickening and/or emulsifying and/or stabilizing agent for preparing cosmetic formulations.
⁽⁵⁾ Sepinov ™ EMT 10 (INCI name:Hydroxyethyl Acrylate/Sodium Acryloyldimethyl Taurate Copolymer is a crosslinked anionic polyelectrolyte used as a thickening and/or emulsifying and/or stabilizing agent for preparing cosmetic formulations.
⁽⁶⁾ Euxyl ™ PE 9010 (INCI name: Phenoxyethanol (and) Ethylhexylglycerin) is a preserving agent used for preparing cosmetic formulations.
⁽⁷⁾ Sensiva ™ PA40 (INCI name: 3-Phenylpropan-1-ol and Octane-1,2-diol and Propane-1,3-diol) is a preserving agent used for preparing cosmetic formulations.

A.1 Transepidermal Water Loss (or TEWL) Measurement.

The transepidermal water loss is measured using a device sold under the brand name Tewameter™ 300 by the company Courage & Khazaka.

The cylindrical measuring chamber of the Tewameter™ 300 device was applied over the insert for which a seal ensured the leak tightness between the measuring cell and the fabric. The value of the water evaporation rate (expressed in g/m²·h) was determined automatically. The environmental conditions during the TEWL (transepidermal water loss) experiments were controlled at a stable temperature of 20° C. and a relative humidity of 50%.

Explants with impaired barrier function (by the tape-stripping method) characterized by a transepidermal water loss of greater than 35 g/m²·h, are treated 1 hour after impairment of the barrier function and every day for five days with a formulation comprising 1% of composition C_{A}. The transepidermal water loss is evaluated on conclusion of these five days of treatment.

The experiments were replicated three times.

The values were expressed as mean±sd [standard deviation].

For each condition, the percentages of effect were calculated as follows:

% effect=[mean(condition)]/[mean(impaired epidermis)]×100−100

Statistical analysis was performed using a Student's t test and a significance level set at 5%, by comparing the conditions in pairs. A difference between the efficiency of two products was considered:
significant if p<0.05;
“at the significance level” if 0.05 s p<0.1;
and not significant if p>0.1.

The results are collated in Table 4 below:

TABLE 4

| | TEWL (g/m² · h) ± SD | | |
|---|---|---|---|
| | D 0 | D 5 | % effect |
| Healthy explants | — | 10.0 ± 1.6 | — |
| Impaired explants | 41.8 ± 3.9 | 36.6 ± 4.2 | −13%^{SL} |
| Impaired explants + | 52.0 ± 3.5 | 37.1 ± 4.5 | −29%*** |
| Formulation (F₁) | | | |
| containing 1% of | | | |
| Composition C_{A} | | | |

Change in transepidermal water loss
SL 0.1 < p < 0.05,
***p < 0.001

The decrease in the transepidermal water loss means an improvement in the skin's barrier function, in other words the skin is better hydrated.

Thus, the treatment of the impaired explants with the combination containing composition C_{A}, which is the subject of the present invention, makes it possible to reduce the transepidermal water loss between D0 and D5, thus indicating a better state of moisturization of the skin.

A.2 Measurement of the Intercellular Spaces

The term “intercellular space” refers to the space between two neighboring cells. Explants with an impaired barrier function (by the “tape-stripping” method) characterized by a transepidermal water loss of greater than 35 g/m²·h, are treated one hour after impairment of the barrier function and every day for five days with a formulation comprising 1% of composition C_{A}.

The experiments were replicated three times.

The intercellular spaces between the keratinocytes are measured via the method consisting of an ultrastructure study by Transmission Electron Microscopy (TEM) using a Hitachi HT7700 microscope.

Tissues were fixed with 2% glutaraldehyde in 0.1 M Sorensen phosphate buffer (pH 7.4), stained with aqueous 2% uranyl acetate solution and then dehydrated and embedded in epoxy resin (Epon 812). Ultrafine sections (50 nm) were mounted on copper grids.

For each condition, the intercellular spaces between the keratinocytes of the basal layer of the epidermis (the deepest layer of the epidermis) were measured on the basis of the images obtained. This analysis was not performed on healthy explants.

The experiments were replicated three times.

The values were expressed as mean±sd [standard deviation].

For each condition, the percentages of effect were calculated as follows:

% effect=[mean(condition)]/[mean(impaired epidermis)]×100−100

Statistical analysis was performed using a Student's t test and a significance level set at 5%, by comparing the conditions in pairs. A difference between the efficiency of two products was considered:
significant if p<0.05;
“at the significance level” if 0.05 s p<0.1;
and not significant if p>0.1.

TABLE 5

| | Intercellular spaces (μm) | |
|---|---|---|
| | Mean ± SD | % effect |
| Impaired explants | 1.15 ± 0.27 | — |
| Impaired explants + | 0.45 ± 0.12 | −61%*** |
| Formulation (F₁) | | |
| containing 1% of | | |
| composition C_{A} | | |

measurement of the intercellular spaces on impaired explants
***p < 0.001

The reduction in the intercellular spaces between the keratinocytes of the basal layer of the epidermis by a value of −61%, after association between the impaired explants and composition C_{A} according to the invention, reflects better cohesion and better organization of the tissue. Being more cohesive and better organized, the explant limits its water loss, which leads to a decrease in the transepidermal water loss and thus an improvement in the skin's barrier function and better skin moisturization.

A.3 Measurement of the Stratum Corneum Thickness

The term “stratum corneum thickness” refers to the thickness of the most superficial layer of the epidermis or cornified layer.

Explants with impaired barrier function (by the tape-stripping method) characterized by a transepidermal water loss of greater than 35 g/m²·h, are treated 1 hour after impairment of the barrier function and every day for five days with a formulation comprising 1% of composition C_{A}.

The experiments were replicated three times.

The stratum corneum thickness was measured via the method consisting of an ultrastructure study by Transmission Electron Microscopy (TEM) using a Hitachi HT7700 microscope.

Tissues were fixed with 2% glutaraldehyde in 0.1 M Sorensen phosphate buffer (pH 7.4), stained with aqueous 2% uranyl acetate solution and then dehydrated and embedded in epoxy resin (Epon 812). Ultrafine sections (50 nm) were mounted on copper grids.

For each condition, the thickness of the stratum corneum was measured on the basis of the images obtained (four measurements per image). This analysis was not performed on healthy explants.

The experiments were replicated three times.

The values were expressed as mean±sd [standard deviation].

For each condition, the percentages of effect were calculated as follows:

% effect=[mean(condition)]/[mean(impaired epidermis)]×100−100

Statistical analysis was performed using a Student's t test and a significance level set at 5%, by comparing the conditions in pairs. A difference between the efficiency of two products was considered:
significant if p<0.05;
“at the significance level” if 0.05 s p<0.1;
and not significant if p>0.1.

TABLE 6

| | Thickness of the stratum | |
|---|---|---|
| | corneum (μm) | |
| | Mean ± SD | % effect |
| Impaired explants | 7.48 ± 1.07 | — |
| Impaired explants + | 13.26 ± 2.33 | 77%*** |
| Formulation (F₁) | | |
| containing 1% of | | |
| composition C_{A} | | |

measurement of the thickness of the stratum corneum on impaired explants
***p < 0.001

The stratum corneum is the most superficial cell layer of the epidermis, the most superficial tissue of the skin.

The increase in the thickness of this layer by +77%, when composition C_{A} according to the invention is combined with impaired explants, makes it possible to limit the water loss, resulting in better moisturization of the skin and improved restoration of the barrier function.

A.4 Measurement of the Biochemical Factors Promoting Skin Moisturization.

The term “NMF” (acronym for “Natural Moisturizing Factor”) refers to a natural moisturizing factor comprising a set of hygroscopic substances located within the corneocytes of the epidermis.

The term “ceramides” refers to chemical compounds of the ceramide family whose function is to maintain the cohesion of the various elements of the skin. Ceramides form a protective layer to help the skin combat dehydration and to protect it against external attack.

Explants with impaired barrier function (by the tape-stripping method) characterized by a transepidermal water loss of greater than 35 g/m²·h, are treated 1 hour after impairment of the barrier function and every day for five days with a formulation comprising 1% of composition C_{A}.

The experiments were replicated twice.

The values were expressed as mean±sd [standard deviation].

For each condition, the percentages of effect were calculated as follows:

% effect=[mean(condition)]/[mean(impaired epidermis)]×100−100

% restoration=100×[mean(composition C_{A})−mean(impaired epidermis)]/[mean(healthy epidermis)−mean(impaired epidermis)].

The NMFs are measured by the LC-MS/MS chromatography method using an Agilent 1100 HPLC system (Agilent Technologies) coupled to a Micromass Quattro Micro API mass spectrometer (Waters), equipped with electrospray ionization. The values obtained from the NMF measurement are expressed in micrograms per milligram of protein.

The ceramides are measured by the LC-MS/MS chromatography method using a system consisting of a high-pressure liquid chromatography or HPLC UltiMate 3000 liquid (ThermoScientific) coupled to an MSQ Plus mass spectrometer (ThermoScientific), equipped with atmospheric pressure chemical ionization (APCI).

The values obtained from the ceramide measurement are expressed in arbitrary units per milligram of protein.

TABLE 7

| | Ceramides | | | | | |
|---|---|---|---|---|---|---|
| | (UA/mg of | | | NMFs (μg/mg | | |
| | prot) | | % | of prot) | | % |
| | Mean ± SD | % effect | restoration | Mean ± SD | % effect | restoration |
| Healthy | 48.56 ± 3.56 | — | — | 54.46 ± 3.85 | — | — |
| explants | | | | | | |
| Impaired | 28.13 ± 2.81 | −42% | — | 29.24 ± 3.43 | −46% | — |
| explants | | vs | | | vs | |
| | | Healthy | | | Healthy | |
| | | explants | | | explants | |
| Impaired | 32.33 ± 2.56 | +15% | 24% | 36.49 ± 2.73 | +25% | 32% |
| explants + | | vs | | | vs | |
| Formulation | | | | | | |
| (F₁) | | Impaired | | | Impaired | |
| containing | | explants | | | explants | |
| 1% of | | | | | | |
| composition | | | | | | |
| C_{A} | | | | | | |

measurement of ceramides and NMFs on untreated impaired explants and on impaired explants treated with composition C_{A} according to the invention (no statistics—n=2)

The restoration of 24% of the amount of ceramides after treatment of the impaired explants with composition C_{A} according to the invention leads to and reflects an improvement in the restoration of the barrier function.

The restoration of 32% of the amount of NMFs after treatment of the impaired explants with composition C_{A} according to the invention leads to and reflects an improvement in the differentiation of the keratinocytes and thus an improvement in the restoration of the skin's barrier function.

The restoration of the skin's barrier function makes it possible to combat dehydration of the skin and to protect it against external attack.

A.5 Measurement of the Number of Keratohyalin Grains

Keratohyalin grains are constituents of the granular layer of the skin.

The increase in the number of keratohyalin grains (containing pro-filaggrin) reflects an improvement in the keratinocyte differentiation process.

Explants with impaired barrier function (by the tape-stripping method) characterized by a transepidermal water loss of greater than 35 g/m²·h, are treated 1 hour after impairment of the barrier function and every day for five days with a formulation comprising 1% of composition C_{A}.

The keratohyalin grains are measured via the method consisting of an ultrastructure study by Transmission Electron Microscopy (TEM) using a Hitachi HT7700 microscope.

Tissues were fixed with 2% glutaraldehyde in 0.1 M Sorensen phosphate buffer (pH 7.4), stained with aqueous 2% uranyl acetate solution and then dehydrated and embedded in epoxy resin (Epon 812). Ultrafine sections (50 nm) were mounted on copper grids.

For each condition, a quantification of the keratohyalin grains was performed on the basis of the images obtained. This analysis was not performed on healthy explants.

The experiments were replicated three times.

The values were expressed as mean±sd [standard deviation].

For each condition, the percentages of effect were calculated as follows:

% effect=[mean(condition)]/[mean(impaired epidermis)]×100−100

Statistical analysis was performed using a Student's t test and a significance level set at 5%, by comparing the conditions in pairs. A difference between the efficiency of two products was considered:
significant if p<0.05;
“at the significance level” if 0.05 s p<0.1;
and not significant if p>0.1.

TABLE 8

| | Number of keratohyalin | |
|---|---|---|
| | grains | |
| | Mean ± SD | % effect |
| Impaired explants | 21.9 ± 4.5 | — |
| Impaired explants + | 32.6 ± 5.6 | 49%*** |
| Formulation (F₁) | | |
| containing 1% of | | |
| composition C_{A} | | |

measurement of the number of keratohyalin grains on the impaired explants treated with composition C_{A} according to the invention
***p < 0.001

The increase in the number of keratohyalin grains when the impaired explants are combined with composition C_{A} that is a subject of the present invention, relative to the number of keratohyalin grains of the impaired explants, reflects an improvement in the keratinocyte differentiation process and consequently an improvement in the keratinocyte differentiation process and thus an improvement in the restoration of the skin's barrier function.

A.6 Analysis of the Lacunar Zones of Human Skin

Analyses were performed in order to study the lacunar zones which have an impact on the moisturizing effect.

The term “lacunar zones” means hydrophilic zones, resulting from the degradation of corneodesmosomes, present in the stratum corneum, containing water and numerous proteins including enzymes involved in the metabolic processing of fats and proteins.

The analysis of the lacunar zones is performed via the method consisting of an ultrastructure study by Transmission Electron Microscopy (TEM) using a Hitachi HT7700 microscope.

Tissues were fixed with 2% glutaraldehyde in 0.1 M Sorensen phosphate buffer (pH 7.4), stained with aqueous 2% uranyl acetate solution and then dehydrated and embedded in epoxy resin (Epon 812). Ultrafine sections (50 nm) were mounted on copper grids.

For each condition, quantification of the lacunar zones was performed on the basis of the images obtained. This analysis was not performed on healthy explants.

The experiments were replicated three times.

The values were expressed as mean±sd [standard deviation].

For each condition, the percentages of effect were calculated as follows:

% effect=[mean(condition)]/[mean(impaired epidermis)]×100−100

Statistical analysis was performed using a Student's t test and a significance level set at 5%, by comparing the conditions in pairs. A difference between the efficiency of two products was considered:
significant if p<0.05;
“at the significance level” if 0.05 s p<0.1;
and not significant if p>0.1.

TABLE 9

| | Number of lacunar zones | |
|---|---|---|
| | Mean ± SD | % effect |
| Impaired explants | 2.7 ± 0.6 | — |
| Impaired explants + | 6.7 ± 1.5 | 150%* |
| Formulation (F₁) | | |
| containing 1% of | | |
| composition C_{A} | | |

measurement of the number of lacunar zones on the impaired explants treated with composition C_{A} according to the invention
*p < 0.05

The impaired explants treated with composition C_{A} according to the invention have a greater number of lacunar zones than the impaired explants not combined with composition C_{A}, reflecting a higher water content in the analyzed skin and thus better moisturization.

A.7 Analysis of Human Skin by Corneometry

The object of corneometry analysis of human skin is to determine the state of moisturization of the upper layers of the epidermis (stratum corneum) by measuring their electrical properties.

This analysis by corneometry is performed via the method consisting in measuring the capacitance of the dielectric medium. Any change in the moisturization of the skin surface causes a modification of these dielectric constants, and consequently of the capacitance measurement. The corneometer measures this capacitance of the most superficial layers of the epidermis, using a probe applied vertically on the skin, with a constant pressure. During the measurement, an electric field penetrates the upper layers of the skin and the dielectric constant is measured. The measurement is obtained 1 second after the application. It is expressed in arbitrary units ranging from 0 to 125 which denote a moisturization index (6-10: very dry skin—10-50: dry skin—50-125: hydrated skin).

The corneometer used in this study was a CM825 Cormeometer® (Courage & Khazaka). Thus, women were recruited to evaluate the moisturizing effect of composition C_{A} versus placebo and versus its glycerol equivalent (0.364%). They applied the formulations (formulation (F₁) described previously and formulation (F′₁) differing from (F₁) only by the absence of composition C_{A}) twice a day for 21 days, with measurements at DO, D7 and D21.

The moisturization was evaluated by corneometry (measurement of the water content of the stratum corneum).
The results are as follows:

TABLE 10

| | Variation of the |
|---|---|
| | moisturization index (AU) |
| Product | between D 21-D 0 |
| Placebo | +32.7% (p < 0.0001) |
| Formulation (F₁) containing | +46.7% (p < 0.0001) |
| 1% of Composition C_{A} | |
| Glycerol 0.364% | +36.1% (p < 0.0001) |
| | Variation of the |
| | moisturization index (AU) |
| Product | vs placebo on D 29 |
| Formulation (F₁) containing | 38.7% (p = 0.0077) |
| 1% of Composition C_{A} | |
| | Variation of the |
| | moisturization index (AU) |
| Product | vs glycerol 0.364% on D 21 |
| Formulation (F₁) containing | 31.65% (p = 0.0397) |
| 1% of Composition C_{A} | |

This study shows the moisturizing capabilities of composition C_{A} according to the invention, after a duration of 21 days.

### General Conclusions

In conclusion, the experimental tests showed that composition C_{A} according to the invention, comprising the lyzate (Ly) of dedifferentiated cells of the plant Helichrysum stoechas, is capable of increasing and/or maintaining the state of moisturization of human skin and/or scalp more particularly dry and/or sensitive and/or reactive skin, by reducing the transepidermal water loss, by decreasing the intercellular spaces between the keratinocytes of the basal layer of the epidermis, by increasing the thickness of the stratum corneum, stimulating the restoration of the cutaneous barrier and by increasing the number of lacunar zones.

### B) Formulations

In the following formulations, the percentages are expressed on the basis of the weight of the formulation.

### B.1 Face Makeup-Removing Fluid

### Formula

| | |
|---|---|
| Composition (C_{A}) | 10.00% |
| Methyl paraben | 0.15% |
| Phenoxyethanol | 0.80% |
| Sepicalm ™ S | 1.00% |
| Perfume/Fragrance | 0.10% |
| Water qs | 100.00% |

### Procedure:

Mix the various ingredients in the water with magnetic stirring, in the order indicated, and adjust the pH to about 7.

B.2 Infant Hair and Body Shampoo

### Formula

A

| | |
|---|---|
| Composition (C_{A}) | 15.00% |
| Proteol ™ APL | 5.00% |
| Sepicide ™ HB | 0.50% |
| Perfume/Fragrance | 0.10% |

B

| | |
|---|---|
| Water | 20.00% |
| Capigel ™ 98 | 3.50% |

C

| | |
|---|---|
| Water qs | 100.00% |
| Sepicide ™ CI | 0.30% |
| Colorant | qs |
| Sodium hydroxide | qs pH = 7.2 |

### Procedure:

Mix composition (C_{A}) with the Proteol™ APL and the Sepicide™ HB (Phase A). Dilute the Capigel™ 98 in a portion of the water and add it to phase A obtained previously (Phase B). Add the rest of the water to phase B, followed by the Sepicide™ CI and the colorant. Adjust the pH of the mixture to about 7.2 with sodium hydroxide.

B.3 Mild Foaming Gel

### Formula

A

| | |
|---|---|
| Composition (C_{A}) | 8.50% |
| Proteol ™ APL | 3.00% |
| Euxyl ™ PE9010 | 1.00% |
| Perfume/Fragrance | 0.10% |

B

| | |
|---|---|
| Water q.s. | 100.00% |
| Lactic acid | qs pH = 6.0 |

### Procedure:

Dissolve the perfume and the preserving agent Euxyl™ PE9010 in the mixture composed of composition C_{A} and the Proteol™ APL (phase A). Add the water and adjust the pH to about 6.0 with lactic acid.

B.4 Frequent-Use Shampoo

### Formula

A

| | |
|---|---|
| Composition (C_{A}) | 12.80% |
| Proteol ™ OAT | 5.00% |
| Euxyl ™ PE9010 | 1.00% |
| Perfume/Fragrance | 0.30% |
| Water qs | 100.00% |

B

| | |
|---|---|
| Montaline ™ C40 | 8.50% |
| Lactic acid | qs pH = 6.0 |

Procedure: Mix all the ingredients of phase A and, after homogenization, add the

Montaline™ C40 and adjust the pH to about 6.0 with lactic acid.

B.5 Baby Cleansing Milk

### Formula

A

| | |
|---|---|
| Simulsol ™ 165 | 2.00% |
| Montanov ™ 202 | 1.00% |
| Lanol ™ 99 | 3.00% |
| Dimethicone | 1.00% |
| Isohexadecane | 3.00% |

B

| | |
|---|---|
| Water qs | 100.00% |

C

| | |
|---|---|
| Sepiplus ™ 400 | 0.30% |

D

| | |
|---|---|
| Composition (C_{A}) | 6.35% |

E

| | |
|---|---|
| Sepicide ™ HB | 0.30% |
| DMDM Hydantoin | 0.20% |
| Perfume/Fragrance | 0.10% |

Procedure: Heat, separately, phases A and B constituted by mixing the various constituents. Add phase C to the hot fatty phase and make the emulsion by pouring in the aqueous phase; homogenize for a few minutes with vigorous stirring (by means of a rotor/stator turbomixer). Next, add phase D to the hot emulsion and cool the emulsion with moderate stirring down to room temperature. Add phase E at 40° C.

B.6 Infant Shower Gel Formula

A

| | |
|---|---|
| Water | 56.06% |
| Sepimax ™ Zen | 3.00% |
| Sepiplus ™ S | 0.80% |

B

| | |
|---|---|
| Proteol ™ OAT | 20.80% |
| Oramix ™ NS 10 | 9.30% |
| Amonyl ™ 265 BA | 5.10% |

C

| | |
|---|---|
| Composition (C_{A}) | 2.00% |
| Glyceryl glucoside | 1.00% |
| Phenoxyethanol & ethylhexyl glycerol | 1.00% |
| Perfume/Fragrance | 0.90% |
| Colorant | 0.04% |

### Procedure:

Disperse the Sepimax™ ZEN in the water and stir using a mechanical stirrer equipped with a deflocculator, a counter-rotating impeller and an anchor paddle, until a perfectly smooth gel is obtained. Add the Sepiplus™ S and then stir until the mixture is homogeneous. Next, add the ingredients of phase B, homogenize and individually add the additives of phase C. Adjust the pH to 6.0-6.5.

B.7 BB Cream

### Formula

A

| | |
|---|---|
| Easynov ™ | 2.30% |
| Lanol ™ 99 | 1.00% |
| Sepimat ™ H10W | 1.00% |
| Ethylhexyl methoxycinnamate | 5.00% |

B

| | |
|---|---|
| Cyclomethicone | 6.00% |
| Triethoxycaprylsilane & alumina-silane & titanium oxide | 8.00% |
| Red iron oxide & triethoxycaprylsilane | 0.24% |
| Yellow iron oxide & triethoxycaprylsilane | 0.66% |
| Black iron oxide & triethoxycaprylsilane | 0.09% |
| Perfume/Fragrance | 0.10% |

C

| | |
|---|---|
| Water | qs 100% |
| Sepinov ™ EMT10 | 1.20% |

D

| | |
|---|---|
| Composition (C_{A}) | 2.00% |
| Sepitonic ™ M3 | 1.00% |
| Phenoxyethanol & ethylhexyl glycerol | 1.00% |

### Procedure:

Prepare phase B by mixing the various ingredients and homogenize using a mixer equipped with a rotor-stator system at a spin speed of 4500 rpm, for a period of 6 minutes. Prepare phase C by adding the Sepinov™ EMT10 to the mixture of water and glycerol, and homogenize using a mixer equipped with a rotor-stator system at a spin speed of 4000 rpm for 4 minutes. Add phases A and B to phase C, and stir the resulting mixture using a mechanical stirrer equipped with an anchor paddle, at a speed of 30 rpm for 2 minutes, and then at a speed of 50 rpm for 20 minutes. Add the components of the phase one by one and stir at a speed of 50 rpm for 25 minutes.

B.8 High-Protection Antisun Spray with an SPF of Greater than 30

### Formula

A

| | |
|---|---|
| Montanov ™ L | 1.00% |
| Montanov ™ 82 | 1.00% |
| C12-15 Alkyl benzoate | 17.00% |
| Dimethicone | 3.00% |
| Octocrylene | 6.00% |
| Ethylhexyl methoxycinnamate | 6.00% |
| Bis(ethylhexyloxyphenol)methoxyphenyltriazine | 3.00% |
| Tocopherol | 0.05% |

B

| | |
|---|---|
| Water | qs 100% |

C

| | |
|---|---|
| Simulgel ™ INS 100 | 0.50% |
| Cyclodimethicone | 5.00% |

D

| | |
|---|---|
| Composition (C_{A}) | 3.00% |
| Phenoxyethanol & ethylhexyl glycerol | 1.00% |
| Perfume/Fragrance | 0.20% |

E

| | |
|---|---|
| Methylenebis(benzotriazolyl)tetramethylbutyl phenol | 10.00% |
| 25% Citric acid | qs pH = 5 |

B.9 Impregnating moisturizing bath for paper masks Formula

B.9 Impregnating moisturizing
bath for paper masks
Formula

| A | |
|---|---|
| Water qs | 100% |
| Glycerol | 2.00% |
| B | |
| Sepimax™ Zen | 0.15% |
| C | |
| Phenoxyethanol and | 1.00% |
| ethylhexylglycerol | |
| 1,2-Hexanediol | 1.00% |
| D | |
| Composition (C_{A}) | 1.00% |

Sepicalm™ S: Mixture of N-cocoylamino acids, sarcosine, potassium aspartate and magnesium aspartate as described in WO 98/09611;

Proteol™ APL: Mixture of sodium salts of N-cocoyl amino acids, obtained by acylation of characteristic amino acids of apple juice;

Sepicide™ HB: Mixture of phenoxyethanol, methyl paraben, ethyl paraben, propyl paraben and butyl paraben, which is a preserving agent;

Capigel™ 98: Copolymer of acrylates;

Sepicide™ CI: Imidazoline urea, is a preservative;

Sepicide™ HB: Mixture of phenoxyethanol, methyl paraben, ethyl paraben, propyl paraben, butyl paraben and isobutyl paraben, is a preserving agent;

Euxyl™ PE9010: Mixture of phenoxyethanol and ethylhexyl glycerol;

Proteol™ OAT: Mixture of N-lauryl amino acids obtained by total hydrolysis of oat protein as described in WO 94/26694;

Montaline™ C40: Monoethanolamine cocamidopropyl betainamide chloride salt;

Simulsol™ 165: Mixture of PEG-100 stearate and glyceryl stearate;

Sepiplus™ 400: Self-invertible inverse latex of polyacrylates in polyisobutene and including polysorbate 20, as described in WO 2005/040230;

Sepimax™ Zen (INCI name: Polyacrylate Crosspolymer-6): Thickening polymer in the form of a powder;

Sepiplus™ S (INCI name: Hydroxyethyl Acrylate/Sodium Acryloyldimethyl Taurate Copolymer & Polyisobutene & PEG-7 Trimethylolpropane Cononut Ether): Self-invertible inverse latex;

Oramix™ NS 10 (INCI name: Decyl glucoside) is a foaming agent used for preparing cosmetic formulations;

Amonyl™ 265 BA (INCI name: Cocobetaine): Foaming amphoteric surfactant;

Sepinov™ EMT10 (INCI name: Hydroxyethyl Acrylate/Sodium Acryloyldimethyl Taurate Copolymer): Thickening copolymer in the form of a powder;

Easynov™ (INCI name: Octyldodecanol and Octyldodecyl Xyloside and PEG-30 Dipolyhydroxystearate): Emulsifying agent having a lipophilic tendency;

Sepimat™ H10 FW (INCI name: Methyl Methacrylate Crosspolymer and Squalane): polymer used as a texture agent;

Sepitonic™ M₃ (INCI name: Magnesium Aspartate and Zinc Gluconate and Copper Gluconate): Mixture used as free-radical scavenger and energizing agent for cells;

Montanov™ L (INCI name: C14-22 Alcohols and C12-20 Alkylglucoside): Emulsifying agent;

Montanov™ 82 (INCI name: Cetearyl Alcohol and Cocoyl glucoside): Emulsifying agent;

Simulgel™ INS100 (INCI name: Hydroxyethyl Acrylate/Sodium Acryloydimethyl Taurate Copolymer and isohexadecane and Polysorbate 60): Polymeric thickener.

1. A lyzate of dedifferentiated cells of the plant Helichrysum stoechas in a form that is suitable for topical administration, for improving the moisturization of the skin and/or the scalp and/or the lips.

2. A method for producing the lyzate as claimed in claim 1, the method comprising high-pressure homogenization of the culture of dedifferentiated cells of the plant Helichrysum stoechas.

3. A composition suitable for topical use which is in the form of a gel and which comprises, per 100% of its mass:
from 95% to 99.5% by mass of the lyzate as defined in claim 1,
from 0.5% to 5% by mass of at least one thickener and/or gelling agent.

4. The composition as claimed in claim 3, wherein the gelling agents and/or thickeners are chosen from polysaccharides, cellulose and cellulose derivatives, starches and linear or branched or crosslinked polymers of polyelectrolyte type.

5. The composition as claimed in claim 3, in a form which is suitable for topical administration, for improving the moisturization of the skin and/or the scalp and/or the lips.

6. A composition suitable for topical use which is in the form of a gel and which comprises, per 100% of its mass:
from 50% to 80% by mass of the lyzate as defined in claim 1,
from 0.1% to 5% by mass of at least one thickener and/or gelling agent, and
from 15% to 49.9% by mass of at least one solvent.

7. The composition as claimed in claim 6, wherein the solvent is chosen from the elements of the group consisting of:
the compounds of formula (Ia):
HO—[CH₂—CH(OH)—CH₂—O]ₙ—H (Ia)
in which n represents an integer greater than or equal to 1 and less than or equal to 15;
the compounds of formula (Ib):
Ra1-C(Rb1)(OH)—C(OH)(Rc1)(Rd1) (Ib),
in which each of the radicals Ra1, Rb1, Rc1 and Rd1 represent, independently of each other, a hydrogen atom or a saturated aliphatic radical including from 1 to 5 carbon atoms, or by the formula (Ib1):
Ra1-C(Rb1)(OH)—[C(Re1)(Rf1)]ₜ—C(OH)(Rc1)(Rd1) (Ib1),
in which t is equal to 1, 2 or 3 and each of the radicals Ra1, Rb1, Re1, Rd1, Re1 and Rf1 represent, independently of each other, a hydrogen atom or a saturated aliphatic radical including from 1 to 5 carbon atoms, it being understood that at least one of the radicals Ra1 or Rb1 and/or at least one of the radicals Rc1 or Rd1 does not represent a hydrogen atom.

8. The composition as claimed in claim 6, wherein the solvent is glycerol.

9. The composition as claimed in claim 6, which is in a form that is suitable for topical administration, for improving moisturization of the skin and/or the scalp and/or the lips.

10. A composition suitable for topical use which is in the form of an emulsion of water-in-oil type or of an emulsion of oil-in-water type, comprising the lyzate as defined in claim 1.

11. A composition suitable for topical use which is in the form of a water-in-oil emulsion and which comprises, per 100% of its mass:
from 60% to 90% by mass of the composition suitable for topical use as defined in claim 3,
from 10% to 40% by mass of a fatty phase comprising i) at least one oil, and and ii) an emulsifying system comprising at least one emulsifying surfactant.

12. The composition as claimed in claim 11, wherein the emulsifying surfactant is chosen from the elements of the group consisting of alkylpolyglycoside compositions, compositions of alkylpolyglycosides and of fatty alcohols, polyglycerol esters, alkoxylated polyglycerol esters, polyglycol polyhydroxystearates, polyglycerol polyhydroxystearates and alkoxylated polyglycerol polyhydroxystearates.

13. A composition suitable for topical use which is in the form of an oil-in-water emulsion and which comprises, per 100% of its mass:
from 50% to 90% by mass of a cosmetically acceptable aqueous phase, said aqueous phase comprising, per 100% of its own mass, from 0.5% to 10% by mass of the lyzate as defined in claim 1,
from 10% to 50% by mass of a fatty phase comprising, per 100% of its own mass:
from 0.5% to 20% by mass of at least one surfactant of oil-in-water type,
from 80% to 99.5% by mass of at least one oil and/or one wax.

14. The composition as claimed in claim 10, which is in a form that is suitable for topical administration, for improving the moisturization of the skin and/or the scalp and/or the lips.

15. The composition as claimed in claim 10, which is in a form that is suitable for topical administration, for reducing and/or eliminating and/or preventing chapping and/or dry patches and/or cracking and/or atopic dermatitis and/or ichthyosis and/or dryness of the skin or mucous membranes, accompanying cutaneous and/or mucosal pathologies.

16. The composition of claim 7, wherein n represents an integer greater than or equal to 1 and less than or equal to 10.

17. The composition of claim 7, wherein n represents an integer greater than or equal to 1 and less than or equal to 6.

18. The composition of claim 7, wherein n represents an integer greater than or equal to 1 and less than or equal to 4.

19. The composition of claim 7, wherein n is 4.

20. The composition of claim 7, wherein n is 4.
